(19) United States
(12) Patent Application Publication
(10) Pub. No.: US 2025/0025185 A1
(43) Pub. Date: Jan. 23, 2025

(54) Surgical Tools and Procedures for Tibial Plateau Leveling Osteotomy

(71) Applicant: Lynch Innovations LLC, Lewes, DE (US)

(72) Inventor: Albert C. Lynch, Landenberg, PA (US)

(21) Appl. No.: 18/660,951

(22) Filed: May 10, 2024

Related U.S. Application Data
(60) Provisional application No. 63/528,081, filed on Jul. 21, 2023.

(51) Int. Cl. A61B 17/15; A61B 17/17; A61B 17/56

(52) U.S. Cl. CPC A61B 17/157; A61B 17/1728; A61B 17/1764; A61B 2017/564

(57) ABSTRACT

Methods and systems related to tibial plateau leveling osteotomy (TPLO) procedures are discussed herein. A disclosed set of TPLO surgical implements includes a surgical guide having a drill guide and an osteotomy guide. The drill guide has a distal guide screw hole and a proximal guide screw hole. The set of surgical implements also includes a bone plate having a distal bone plate screw hole and a proximal bone plate screw hole. The drill guide and the bone plate are complementary in that the distal guide screw hole and the proximal guide screw hole are aligned with a planned pre-operative orientation of a planned distal bone screw location and a planned proximal bone screw location, and the distal bone plate screw hole and the proximal bone plate screw hole are aligned with a planned post-operative orientation of the planned distal bone screw location and the planned proximal bone screw location.

### CROSS REFERENCE TO RELATED APPLICATIONS

This application claims the benefit of U.S. Provisional Patent Application No. 63/528,081, filed Jul. 21, 2023, which is incorporated by reference herein in its entirety for all purposes.

### BACKGROUND

Cranial cruciate ligament (CCL) tear, equivalent to tears of the human anterior cruciate ligament (ACL), is one of the most common surgical conditions in veterinary orthopedics. A CCL tear leads to joint instability and progressive degenerative joint disease, as well as an elevated risk of subsequent meniscal pathology. The tibial plateau leveling osteotomy (TPLO) is considered the gold standard for treatment of CCL disease in veterinary patients.

The TPLO procedure utilizes a radial saw blade to create an osteotomy in the proximal tibia and subsequently rotate the tibial plateau to neutralize cranial tibial thrust, while minimizing strain on the caudal cruciate ligament. As used herein the term “proximal tibia” is used in the standard sense of mammalian anatomy to refer to the upper portion of the tibia where it widens to help form the knee joint. However, the term “proximal” when used to refer to a bone “segment” refers to a post-osteotomy segment of bone that is closest to the knee joint which is in contradistinction to the “distal” bone segment which is the post-osteotomy segment of bone that is on the other side of the osteotomy and relatively distal to the knee joint.

TPLO procedures have exacting guidelines and are generally conducted by specialist surgeons. For example, while executing the radial osteotomy, it is important that the saw blade be oriented parallel to the proximal tibial joint surface, perpendicular to the sagittal plane and with the center of rotation for the radial blade centered over the intercondylar eminences, which represent the biomechanical center of the stifle joint. Additional guidelines, related to the execution of a radial osteotomy, include preservation of sufficient bone at the level of the tibial tuberosity and producing a post-osteotomy proximal tibial bone segment footprint just sufficient to accommodate the “head” of a TPLO bone plate and associated screws, while avoiding placing those screws in the osteotomy or intra-articularly. Failure to meet the exacting guidelines of a TPLO procedure may result in, among other possible complications, limb malalignment, inaccurate alteration of the tibial plateau angle, accelerated joint pathology, tibial fractures, poor implant placement, or implant failure.

Performing an accurate radial osteotomy is dependent on precise execution of the planned osteotomy at the time of surgery, at the intended location and intended trajectory. This is one of the most technically difficult aspects of the procedure, partly due to the triangular cross-section and irregular surface contour of the proximal tibia and the tendency for the oscillating radial saw blade to “skip” along the tibial cortex during initiation of the osteotomy. Approximately fifty procedures, as the primary surgeon performing the osteotomy, are required before acceptable proficiency in performing the osteotomy is attained.

Accurate osteotomies for a TPLO procedure require centering the radial osteotomy on the intercondylar eminences of the tibia. Industry standard procedures have identified specific points with reference to the intercondylar eminences, which are commonly referred to as D1, D2, and D3, which are used to properly center the radial osteotomy. The points were devised as measurements that could be measured on pre-operative radiographs for planning the osteotomy and that could be measured again at the time of surgery to properly center the radial blade with reference to the intercondylar eminences.

Performing the osteotomy is also not the only technically demanding aspect of a TPLO procedure. Additional technically demanding aspects of a TPLO procedure include precision rotation of the post-osteotomy bone segments, achieving temporary stabilization of the post-rotation bone segments, and positioning of the bone plate and screws used for final stabilization. These steps are generally aided using specialized surgical implements such as pins, jigs, and guides. However, available surgical implements for TPLO procedures and the associated procedures facilitated thereby are associated with various drawbacks as described below.

In common TPLO procedures, rotation of the post-osteotomy bone segments and temporary stabilization of the post-rotation bone segments are achieved using a rotation pin and stick pin. After completion of the osteotomy, a rotation pin is placed within the proximal tibial bone segment at an oblique angle to serve as a handle to facilitate rotation of the proximal tibial bone segment. The diameter of this rotation pin is frequently near the core diameter of the screws used in the proximal tibial bone segment to secure the bone plate. The rotation pin therefore results in reduced screw purchase due to the large void in the proximal tibial bone segment once the pin is removed. Additionally, excessive force exerted during rotation may bend the pin or fracture the proximal tibial bone segment. Once the desired amount of bone segment rotation is achieved, the two bone segments are stabilized with a smaller stick pin that engages both segments. The stick pin must be introduced at a specific anatomic location, otherwise it could weaken the bone beneath the patellar tendon leading to fracture of the bone. The stick pin must have a trajectory near parallel to the joint surface to maximize its security of the bone segments, avoid intra-articular trauma and prevent obscuring the desired path of a potential bone screw. Interference with the stick pin may cause the deflection of a locking screw for a bone plate which will significantly affect the strength of the stabilization provided by such a bone plate.

Final stabilization of the bone segments includes additional technical hurdles. When placing the bone plate and screws for final stabilization of the bone segments, it is important that the screws within the proximal bone segment avoid the joint surface and the osteotomy site and that the bone screws in the distal segment remain centered on the bone. It is also important that the osteotomy be well apposed between the proximal and distal tibial segments, as a “gap” may negatively impact healing and result in excessive strain on the bone plate and screws. The cranial (anterior) aspect of the osteotomy tends to gap open despite the stick pin, and a common technique to address this involves the use of forceps engaging both bone segments to apply force across the gap. This can affect the positioning of the TPLO plate on the medial tibial cortex, which may lead to implant failure or tibial fracture.

In addition to specialized procedures using stick pins and rotation pins, TPLO jigs and guides have also been developed to assist in the placement of the osteotomy and the rotation and appropriate temporary stabilization of the bone segments in a TPLO procedure. However, as mentioned previously, available implements for these purposes include certain drawbacks.

The initial TPLO jig described by Barclay Slocum required the placement of pins, parallel to the tibial plateau and perpendicular to the sagittal plane of the tibia, in the distal tibial bone segment and the proximal tibial bone segment. The pin in the proximal tibia segment is placed approximately 4 mm from the joint surface. These pins function as visual references for the desired trajectory of the radial osteotomy, if inserted accurately. These pins are attached to a jig, which serves to maintain orientation of the post-osteotomy proximal bone segment and distal tibial bone segment during the application of a bone plate for definitive fixation. This jig may also serve as a point of mechanical leverage during rotation of the two tibial bone segments, if a second pin is placed in the proximal tibial bone segment. The proximal insertion site of the proximal jig pin is required to permit the radial osteotomy; however, its physical presence, in association with the attached jig, may affect the surgeon's accuracy for osteotomy positioning and trajectory. Additionally, even in the hands of experienced TPLO surgeons, there is an approximately 5% risk of intra-articular jig pin placement, which may lead to serious complications and joint pathology.

Following the development of the Barclay Slocum jig, a radial saw guide attachment was developed that can be attached to a modified Slocum style TPLO jig and assist the positioning of the osteotomy. However, reliable accuracy has not been demonstrated with this system as approximately 70% of the executed osteotomies using this attachment were distal to their intended location on the tibial cortex. In other words, the executed osteotomies had an improper distance of eccentricity (i.e., the blade's rotational pivot point was not at the intercondylar eminences of the bone).

A concentric saw guide has also been developed, with an intended function and structure like a radial saw guide but affording unilateral support on the convex surface of the saw blade, opposed to the concave surface as the radial guide system does. However, the only demonstrated benefit of this system was to reduce saw blade “chatter” on replica bone models.

Patient specific guides developed using computed tomographic (CT) imaging, computer modeling, and 3D printing have been documented that precisely reflect the surface topographic anatomy specific to a single patient, with the goal to function as an osteotomy guide or a drill guide for pin or bone screw placement. Unfortunately, the requirement for CT imaging, computer modeling, and 3D printing adds unacceptable time, radiation exposure, cost, and specificity to a single patient, which results in minimal practical utilization of this type of system.

Bone plates, utilized for stabilization of the tibial bone segments in their desired end alignment state post-rotation, have been specifically developed for the TPLO procedure. These TPLO plates commonly have a “head”, which will engage the post-osteotomy proximal tibial bone segment via bone screws, and a “stem”, which will engage the distal tibial bone segment. Additionally, the TPLO plate may have a profile that reflects the topographic conformation of the proximal medial tibial cortex, where it is to be placed, and utilize locking and/or non-locking bone screws. These locking bone screws form a rigid construct when “locked” into the TPLO plate, thereby minimizing the need for contouring of the TPLO plate that would be necessary with a non-locking bone screw to bone plate construct. Locking screws may be fixed angle, only engaging the plate at one specific pre-established angle trajectory, or poly-axial, able to lock into the bone plate within a range of acceptable angulation of the screw trajectory. The trajectory of fixed angle locking screws within the head of TPLO plates is commonly developed to angle the bone screw away from the joint surface and away from the osteotomy. The trajectory of fixed angle locking screws within the stem of TPLO plates is perpendicular to the surface of the bone plate. The stem of the TPLO plate may be straight or curved, depending on the manufacturer.

TPLO procedures have a long-established and proven tendency to produce beneficial patient outcomes when executed properly. However, given the complexity of the procedure and the state of the art in surgical implements for assisting in the conduct of a TPLO procedure, there is an insufficient number of surgeons capable of safely and reliably executing TPLO procedures. Additionally, there is currently an unacceptable level of inter and intra-surgeon variability regarding the precision of executed osteotomies, the magnitude of purely sagittal plane rotation, and the precision of bone plate placement. Advances in surgical implements for TPLO procedures are therefore important for allowing additional surgeons to meet patient demand and to increase the percentage of successfully conducted operations generally.

### SUMMARY

Methods and systems related to tibial plateau leveling osteotomy (TPLO) procedures are discussed herein. Various surgical implements for TPLO procedures are disclosed herein. The implements include surgical guides, bone plates, and other surgical implements. The implements can be conformal but not patient specific. As used herein, the term “conformal” refers to the state of being shaped to be anatomically specific as opposed to being patient specific. The surgical guides can include drill guides, osteotomy guides, and integrated drill and osteotomy guides. The implements can include bone plates, system positioners such as positioner implements, implements for assisting in the translation of the bone segments such as handles which attach to the bone plates, alignment pins, drill sleeves, and other implements as described below. The implements can be organized into libraries of implements that are available to be selected during pre-operative planning or during an operation based on measurements taken during surgery or in response to modifications decided upon by the surgeon in the operating room. Pre-operative and intra-operative surgical methods for TPLO procedures that utilize such implements are also disclosed here.

The implements and associated methods disclosed herein improve upon the state of the art in TPLO surgical implements and address the drawbacks described in the background above. For example, various surgical implements disclosed herein offer, in contrast to systems described in the background above, an integrated osteotomy and drill guide, a system positioner that does not rely on positioning with respect to the D1-3 points as in traditional systems, the ability to incrementally customize the magnitude of rotation of the post-osteotomy bone segments to fit a given patient's requirements, the ability to make intra-operative alterations to the trajectory of the osteotomy, bone screws, or pins, assistance with bone segment rotation, and avoidance of significant pin defects in tibial bone segments. Additionally, specific surgical implements disclosed herein referred to as offset plates, and associated procedures, provide the ability to achieve numerous end magnitude bone rotations using the same screw holes to allow for alterations to the tibial plateau rotation based on intra-operative findings. Various surgical implements and associated procedures disclosed herein are capable of improving outcomes associated with critical steps of the TPLO procedure in the hands of both experienced TPLO surgeons and clinicians without prior TPLO surgical experience. Furthermore, associated procedures disclosed herein have fewer steps than traditional TPLO procedures, provide fewer channels for the introduction of user error, afford a greater degree of flexibility for intra-operative alterations, and do not require the pre-operative generation of patient specific guides.

Surgical procedures associated with specific embodiments of the surgical implements disclosed herein, can be conducted with traditional imaging, and do not require modifications to traditional pre-operative planning, thereby minimizing the amount of training required to certify surgeons to utilize the implements. A TPLO procedure conducted in accordance with this disclosure can utilize planar radiography, which is the current standard for pre-operative TPLO planning. Therefore, there are no additional pre-operative diagnostic imaging requirements with these embodiments. A two-dimensional (2D) template that is specific to these embodiments may be utilized with any conventional radiograph viewing software. In specific embodiments of the invention, the pre-operative planning is unchanged from current TPLO procedure: measure the patient's tibial plateau angle (TPA) and magnitude of required alteration of that TPA to have an end TPA of five degrees, determine the ideal TPLO radial saw blade size, determine the ideal location to initiate the radial osteotomy on the proximal medial tibial cortex, and select an appropriate TPLO bone plate for final stabilization of the tibial bone segments. Accordingly, these surgical procedures allow for less experienced practitioners to achieve more accurate results using the same imaging technology that is used in standard procedures.

Surgical implements disclosed herein can be conformal without being patient specific. For example, a surgical guide could include a conforming bone facing surface and a blade facing surface with an osteotomy guide. The conformal bone facing surface could conform to a standard tibial surface while not being patient specific. The surgical guide could also include portions which are not conformal and can include cut outs at portions that have greater variation from patient to patient. For example, the surgical guide could include a conformal surface for a standard tibial surface that is proximal to the joint while not being flush with the tibial cortex at a distal location on the tibial surface. As another example, the surgical guide could have a cut out at a standard popliteus muscle location. Accordingly, the surgical implements can be used for a broad array of patients and can be selected from a library of potential surgical implements where the library has a reasonable number of elements and fully customized implements are not required. Furthermore, the surgical implements can be reusable to further reduce costs. In specific embodiments where the surgical implements are selected from libraries of implements based on the patient's TPA and the desired radius of the osteotomy, having a limited number of elements in the library for patient-specific sizing in terms of conformity to the bone surface is particularly beneficial as the library already includes a diverse number of elements to accommodate different TPAs and desired radii.

Surgical implements disclosed herein can include drill guides and bone plates which are complementary to assure accurate rotation to a desired degree and correct positioning of the post-operative bone segments in accordance with the desired modification of the patient's TPA. The desired modification of the patient's TPA can be expressed as the difference between the pre-operative TPA and the post-operative TPA. In general, the TPLO procedure targets a final TPA of 5 degrees. Drill guides in accordance with this disclosure can include screw holes that can be used to make at least one proximate screw hole and one distal screw hole in the patient's bone prior to rotation of the osteotomy. The drill guides can be configured to define a pre-operative vector between the screw holes in the bone. Bone plates in accordance with this disclosure can include screw holes which are complementary to the drill guides in that they include bone plate screw holes that define a desired post-operative vector between the screw holes in the bone. The post-operative vector can be a characteristic of the complementary nature of the locations of the screw holes in the drill guide and the locations of the screw holes in the bone plate. Accordingly, the surgical implements can allow for accurate rotation of the bone segments after an osteotomy dependent only upon the appropriate placement of the surgical guide on the patient's bone and attachment of the bone plate to the bone using the screw holes in the bone formed with the help of the surgical guide. Various implements and approaches to achieve these results are disclosed in the detailed description below.

### BRIEF DESCRIPTION OF THE DRAWINGS

The accompanying drawings illustrate various embodiments of systems, methods, and embodiments of various other aspects of the disclosure. A person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates four views of a TPLO procedure that is in accordance with specific embodiments of the inventions disclosed herein.

FIG. 2 illustrates an osteotomy guide with a proximal drill guide that translates with respect to the osteotomy guide and a fixed distal drill guide in accordance with specific embodiments of the invention disclosed herein.

FIG. 3 illustrates a drill guide that translates relative to an osteotomy guide and that includes both a proximal and distal drill guide in accordance with specific embodiments of the invention disclosed herein.

FIG. 4 illustrates an example of a surgical guide in the form of a unibody element which includes a distal drill guide, a proximal drill guide, and an osteotomy guide in accordance with specific embodiments of the inventions disclosed herein.

FIG. 5 illustrates basic imaging images in the form of a mediolateral radiograph and craniocaudal radiograph that can be used for pre-operative planning in accordance with specific embodiments of the inventions disclosed herein.

FIG. 6 illustrates a surgical guide with an integrated drill guide and osteotomy guide that is labeled with markings for the surgical conditions it is designed for in accordance with specific embodiments of the inventions disclosed herein.

FIG. 7 illustrates the application of a conformal but not-patient specific surgical implement to the bone of a patient in accordance with specific embodiments of the inventions disclosed herein.

FIG. 8 illustrates positioner implements for surgical guides that are in accordance with specific embodiments of the inventions disclosed herein.

FIG. 9 illustrates a process for placing a surgical implement on a patient's bone using pins in accordance with specific embodiments of the inventions disclosed herein.

FIG. 10 illustrates a drill sleeve inserted into a screw hole of a drill guide of a surgical guide that is in accordance with specific embodiments of the inventions disclosed herein.

FIG. 11 illustrates a surgical implement for assisting with the translation of a patient bone using an attached bone plate in the form of a nonrigid connector interface and a lever tool in accordance with specific embodiments of the inventions disclosed herein.

FIG. 12 illustrates a surgical implement for assisting with the translation of a patient bone using an attached bone plate in the form of an attachable handle in accordance with specific embodiments of the inventions disclosed herein.

FIG. 13 illustrates a specific method for introducing a bias bone compression and a specific desired result of a biased bone compression in accordance with specific embodiments of the invention disclosed herein.

FIG. 14 illustrates a misalignment between a screw hole formed by a drill guide and a screw hole of a bone plate as used for introducing a bias bone compression in accordance with specific embodiments of the invention disclosed herein.

FIG. 15 illustrates the three views of a surgical guide with adjustment screws to modify the trajectory of a drill guide and osteotomy guide on the surgical guide in accordance with specific embodiments of the inventions disclosed herein.

FIG. 16 illustrates offset plates that can be used in accordance with specific embodiments of the inventions disclosed herein.

FIG. 17 illustrates a flow chart of a set of surgical methods for a TPLO that are in accordance with embodiments of the inventions disclosed herein.

### DESCRIPTION

Reference will now be made in detail to implementations and embodiments of various aspects and variations of systems and methods described herein. Although several exemplary variations of the systems and methods are described herein, other variations of the systems and methods may include aspects of the systems and methods described herein combined in any suitable manner having combinations of all or some of the aspects described.

Methods and systems for tibial plateau leveling osteotomy (TPLO) procedures in accordance with the summary above are disclosed in detail herein. The methods and systems disclosed in this section are nonlimiting embodiments of the invention, are provided for explanatory purposes only, and should not be used to constrict the full scope of the invention. It is to be understood that the disclosed embodiments may or may not overlap with each other. Thus, part of one embodiment, or specific embodiments thereof, may or may not fall within the ambit of another, or specific embodiments thereof, and vice versa. Different embodiments from different aspects may be combined or practiced separately. Many different combinations and sub-combinations of the representative embodiments shown within the broad framework of this invention, that may be apparent to those skilled in the art but not explicitly shown or described, should not be construed as precluded.

Specific embodiments of the invention involve using drill guides and bone plates where the positions of the screw holes in the drill guides and bone plates are complementary. In these embodiments, when screw holes are made in the bone using the screw holes in the drill guide, and the bone is rotated according to a desired TPA adjustment, the screw holes in the bone plate will align with the screw holes in the bone. The drill guide can be used to make a set of holes in the bone after it has been fixed to the bone such as by using stabilization pins.

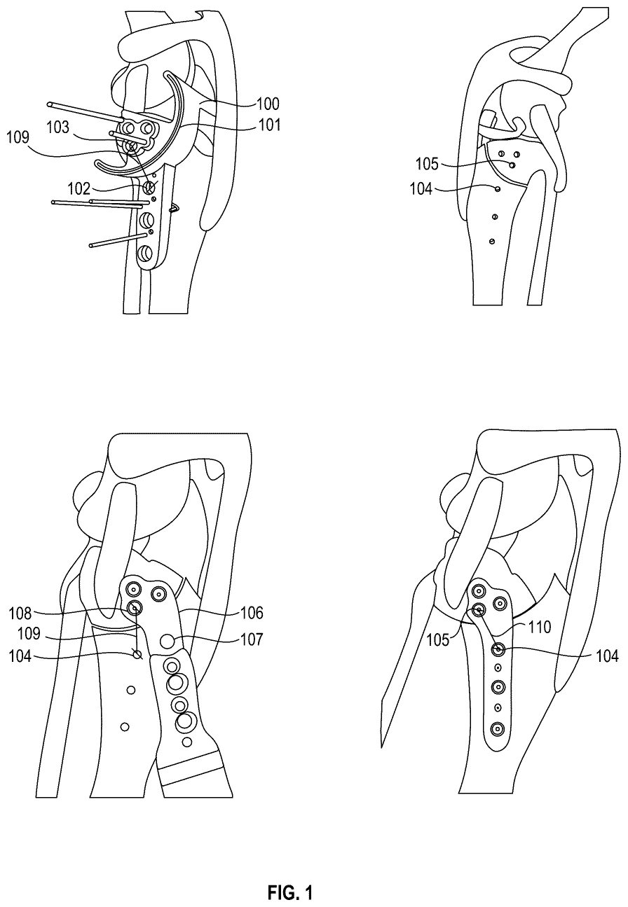

FIG. 1 provides four views of a TPLO procedure that is in accordance with specific embodiments of the inventions disclosed herein. The views include a surgical guide 100 having both a drill guide and an osteotomy guide in a single unibody element. In alternative embodiments, as described below, the drill guide and osteotomy guide may be separate pieces. The osteotomy guide defines a potential site for a TPLO 101. The potential site is an arc that is spaced apart from the joint and is centered on the intercondylar eminences of the bone. The drill guide has a distal guide screw hole 102 and a proximal guide screw hole 103.

FIG. 1 also illustrates a set of screw holes that have been formed in the patient's bone. The screw holes are most clearly visible in the top right view of FIG. 1 in which the osteotomy has been performed and the bone has been flipped over to view its lateral aspect. The screw holes can be formed prior to conducting the osteotomy such that the bone will be sturdier and the screw holes can be formed with a higher degree of precision. The screw holes include two screw holes formed at a planned distal bone screw location 104 and a planned proximal bone screw location 105. The terms proximal and distal are being used here to refer to locations proximal or distal to the joint relative to the location of the osteotomy.

FIG. 1 also illustrates a bone plate 106 having a distal bone plate screw hole 107 and a proximal bone plate screw hole 108. In the bottom left view of FIG. 1, the bone plate has been attached to the proximal post-osteotomy section of the bone using bone screws such as the screw that has been inserted into proximal bone plate screw hole 108 at planned proximal bone screw location 105. In specific embodiments of the invention, the bone plate is then used as a lever to rotate the bone appropriately and adjust the TPA of the patient. In the bottom right view of FIG. 1, the surgeon has used the bone plate to rotate the bone accordingly and the bone plate has been attached to the distal post-osteotomy section of the bone using bone screws such as the screw that has been inserted into distal bone plate screw hole 107 at planned distal bone screw location 104.

As is illustrated by the example in FIG. 1, the drill guide 100 and the bone plate 106 are complementary in that the distal guide screw hole 102 and the proximal guide screw hole 103 are aligned with a planned pre-operative orientation 109 of a planned distal bone screw location 104 and a planned proximal bone screw location 105, and the distal bone plate screw hole 107 and the proximal bone plate screw hole 108 are aligned with a planned post-operative orientation 110 of the planned distal bone screw location 104 and the planned proximal bone screw location 105. Accordingly, so long as the drill guide (e.g., drill guide 100) has been properly placed, assuring the appropriate translation of the bone is simplified to assuring that the distal screw holes of the bone plate are aligned with the distal screw holes in the bone. In other words, assuring the appropriate translation of the bone is simplified to attaching the bone plate via the screw holes formed in the bone using the drill guide.

Methods in accordance with this disclosure include marking a patient's bone at planned screw hole locations. As used herein, the term “marking” can refer to the actual drilling of a screw hole and can also refer to making a mark or indentation for a screw hole to be formed later. The methods comprise contacting a tibia with an osteotomy guide, whereby the osteotomy guide defines a potential site for the tibial plateau leveling osteotomy. The methods also comprise marking a first side of the potential site for a first screw hole using a drill guide that is attached to the osteotomy guide. The method also comprises marking a second side of the potential site for a second screw hole using a drill guide that is attached to the osteotomy guide. The two drill guides can be a proximal drill guide and a distal drill guide. The drill guides can each be either fixedly or detachably attached to the osteotomy guide. As used herein, the two drill guides can be referred to as a “drill guide” either independently or in combination depending on the context.

The methods also comprises cutting the tibia at a potential osteotomy site using an osteotomy guide, aligning a proximal portion of the tibia and a distal portion of the tibia so that the first screw hole and the second screw hole are aligned with a pair of screw holes on a bone plate, and attaching the bone plate using a first screw inserted into the first screw hole and a second screw inserted into the second screw hole. The reason the screw holes in the bone plate will align with the screw holes in the bone when the bone segments have been appropriately rotated is that the drill guide determines the TPA adjustment. In specific embodiments, the drill guide can be selected from a library of drill guides with different TPA adjustments in mind. In alternative embodiments, the drill guide can be adjustable so that a surgeon can select a desired TPA adjustment for the drill guide to implement. When the drill guide is adjustable it can be fixed temporarily in a complementary configuration to the bone plate for which it will be used to form screw holes in the bone.

In specific embodiments, in which the surgical implements include both an osteotomy guide and a drill guide, the drill guide may be detachable or in a fixed position relative to the osteotomy guide. The drill guide could be attached in a manner that allowed the drill guide to translate its position relative to the osteotomy guide while staying attached to the osteotomy guide. The drill guide could translate along the functional axis of rotation of the osteotomy guide. This would allow the surgeon to premeditate the precise rotational offset between the head and stem of the plate required to achieve an end TPA (e.g., of five degrees) once those drill holes have been aligned and secured to the TPLO plate that the drill guide was intended for, based on that individual patient's pre-operative TPA. Therefore, this system is reusable and able to be functionally modified to a specific patient's needs. For example, a surgical guide for a TPLO may comprise an osteotomy guide forming an arc for the TPLO, and a drill guide attached to the osteotomy guide and configured to rotate along the arc.

In specific embodiments, the drill guide can be translated relative to the osteotomy guide in various ways. For example, the translation along the osteotomy guide by the drill guide may be a “slider” plate drill guide. This slider plate guide may articulate with a designed groove, osteotomy saw blade slot, or straddle the outer walls of the osteotomy saw guide. The translation may be achieved by a surgeon pushing the drill guide and then fastening the drill guide in place temporarily using a mechanism of the surgical implement. The translation may alternatively be achieved through an adjustable drill guide which sweeps through the arc of the osteotomy guide according to one or more mechanical gears which push the drill guide along the arc and lock the drill guide in place when the gears are not moving. Temporary stabilization of the translating drill guide in its desired position may be achieved in various ways, such as by using a set screw or pins. Once the drill guide is temporarily stabilized it can be used to insert pins, make marks on the bone segments to identify potential bone screw sites, or to drill holes for bone screw sites.

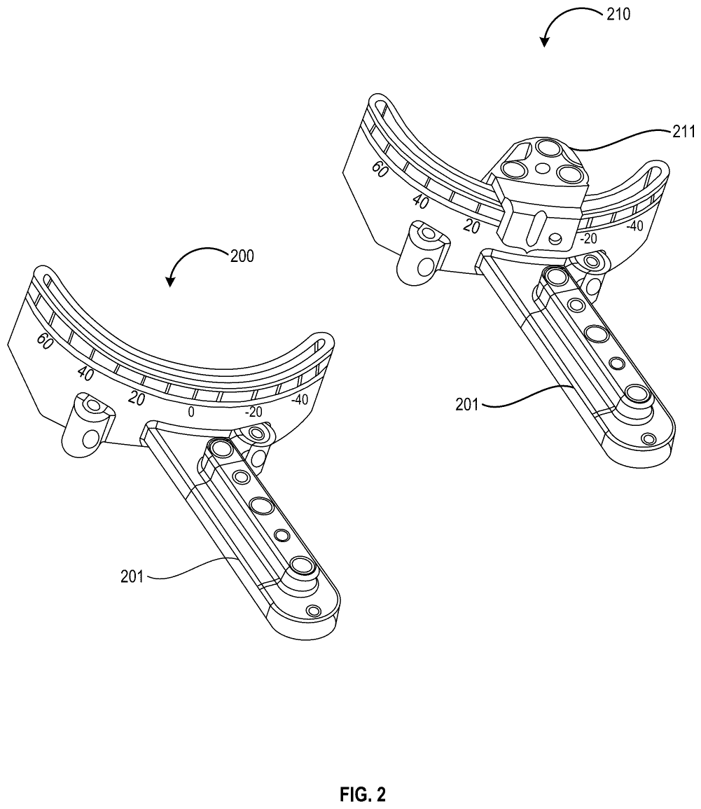

FIG. 2 illustrates an osteotomy guide with a proximal drill guide that is capable of translating with respect to the osteotomy guide and a fixed distal drill guide in accordance with specific embodiments of the invention disclosed herein. As shown, the surgical implement is an integrated osteotomy guide and drill guide. The drill guide is a fully enclosed radial guide. View 200 illustrates osteotomy guide 201 alone while view 210 illustrates osteotomy guide 201 with proximal drill guide 211 attached. The distal drill guide can have the same bone facing contour as the stem of a bone plate that will ultimately be attached to the patient's bone, while the proximal drill guide can have the same bone facing contour as the head of that bone plate.

In specific embodiments, the osteotomy guide or drill guide can include a set of markings where the markings indicate a set of offset angles along the arc through which the drill guide can be translated. An example of such markings is shown in FIG. 2. In specific embodiments, the guidance system may display the magnitude of the offset between the drill guide and a neutral position as the drill guide is translated along the osteotomy guide. For example, this magnitude may be displayed as the degrees of TPA alteration and/or chord length of bone segment rotation that will be introduced after the TPLO. The markings could be formed by shaped lettering on the drill guide or osteotomy guide, by laser or chemical branding, or by some other form of permanent marking.

In specific embodiments of the invention, the drill guide could span the osteotomy guide and include a distal drill guide and a proximal drill guide. Using such a surgical implement, pins or marks could be applied on one side of the osteotomy or drill holes could be formed in the bone using one of the drill guides, the drill guide could be translated along the arc of the osteotomy guide by the pre-operatively determined TPA, and then another set of marks or pins could be applied on the other side of the osteotomy. After that the osteotomy could be performed with both the proximal bone segment and the distal bone segment being marked for alignment with a bone plate via the marks or pins on the two bone segments.

An example where the drill guide that translates relative to the osteotomy guide includes both a proximal and distal drill guide is presented immediately below.

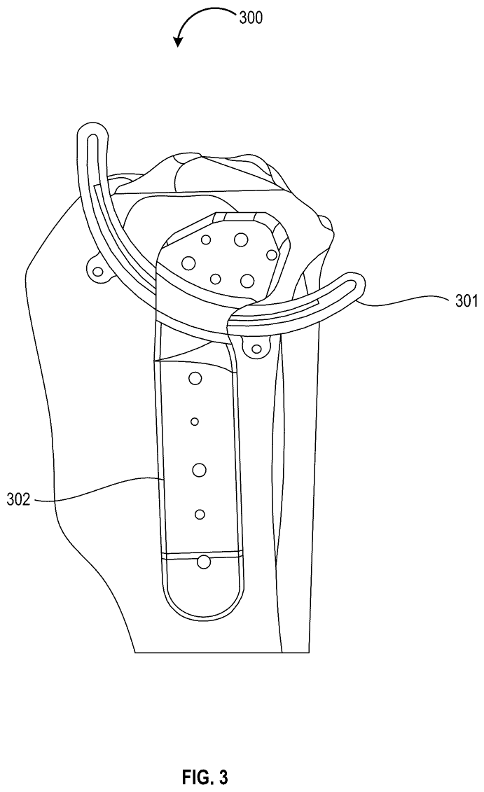

FIG. 3 illustrates a drill guide that translates relative to an osteotomy guide and that includes both a proximal and distal drill guide in accordance with specific embodiments of the invention disclosed herein. View 300 illustrates osteotomy guide 301 with a detachable attached drill guide 302. As illustrated, drill guide 302 includes both a proximal drill guide and a distal drill guide in a unibody element. The drill guide could be used by first marking proximal or distal screw hole locations in the bone, translating the drill guide along the arch of the osteotomy guide, and then marking the alternative screw hole locations.

In specific embodiments of the invention, a drill guide is attached to an osteotomy guide and is configured to rotate along the arc. Such a drill guide could be a distal drill guide while a proximal drill guide could be fixed relative to the osteotomy guide. In alternative embodiments of the invention, the drill guide that is attached to the osteotomy guide and is configured to rotate along the arc could be a proximal drill guide and a distal drill guide could be fixed relative to the osteotomy guide. In these approaches, once the drill guide was placed on the tibia, the fixed drill guide could be used to place pins or marks on one bone segment and the other, adjustable, drill guide could be translated relative to a neutral position by the TPA and then be used to place pins or marks on the other bone segment or to form drill holes in the bone. For the avoidance of doubt, the term bone segment is being used in this context to refer to portions of bones that will ultimately be separate segments once the osteotomy is performed, but in this embodiment the marks are made, or the pins are inserted, before the osteotomy is performed. An example of the drill guide that can translate along the arc of the osteotomy guide being a proximal drill guide was presented in FIG. 2 above.

In specific embodiments, the drill guide can translate between fixed positions relative to the osteotomy guide by being set into locked positions (e.g., fixed angles relative to the osteotomy guide). In alternative embodiments, the drill guide can translate smoothly between various positions relative to the osteotomy guide. In embodiments in which the drill guide can translate between fixed positions, such as lock points which temporarily fix the drill guide in a given position relative to the osteotomy guide, bone plates with heads at different offsets relative to the body of the bone plate can be used to provide a finer matching between the desired TPA and that provided by the disclosed implements as will be described below. The fixed angles may be spaced apart by various amounts (e.g., 5 degrees). The use of fixed angles may further reduce the potential operator error that could be introduced while “setting” an angle on the drill guide particularly for situations in which the cut guide is small.

In specific embodiments, the surgical guide can be selected from a library of surgical guides for a desired TPA adjustment. The entries in the library can differ in terms of the placement of the screw holes in the drill guide of the surgical guide. For example, the entries can differ in an angle around an arc of an osteotomy guide at which the screw holes of the drill guide are positioned. In specific embodiments, the entries in the library can be spaced apart by degree increments in terms of the TPA they are adjusting for. For example, the spacing can be 2.5 degrees, and the TPA they are adjusting for can range from 15 to 30 degrees of TPA.

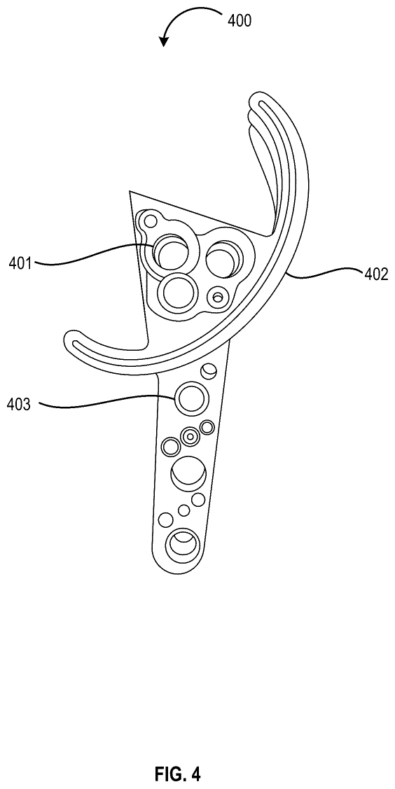

FIG. 4 illustrates an example of a surgical guide 400 in the form of a unibody element which includes a distal drill guide 403, a proximal drill guide 401, and an osteotomy guide 402 in accordance with specific embodiments of the inventions disclosed herein. As illustrated, the proximal drill guide 401 is located at an offset angle along the arc of the osteotomy guide 402. In specific embodiments, the surgical guide can be selected from a library based on that offset angle and the other surgical guides in the library can be identical except for a change in the offset angle of the proximal drill guide 401 along the arc of the osteotomy guide 402. Accordingly, a surgeon can select a surgical guide from the library to impart a desired post-operative TPA to the patient bone.

As stated in the summary, in specific embodiments of the invention, basic imaging can be used for pre-operative planning. The basic imaging can be used to obtain a measurement of the required modification to the TPA and to either determine which surgical guide to select from a library of surgical guides with fixed drill guides, or how to operate a surgical guide with a moveable drill guide to place the screw holes at the desired locations for a desired TPA adjustment. Pre-operative planning can include measuring a TPA in a standard imaging image such as a mediolateral radiograph. Pre-operative planning with basic imaging can also be used to select the desired radial saw blade size. The radial saw blade size may differ depending upon the size of the patient (e.g., breed of a dog). The radial saw blade size can also be selected using the mediolateral radiograph by measuring a desired radius of the arc for the potential osteotomy site. As such, the surgical guides disclosed herein, both those with rotatable drill guides and those with fixed drill guides, can be selected from a library with the entries in the library differing in terms of the size of the blade guide. Pre-operative planning with basic imaging can also be used to select lengths for the bone screws that will be used to secure the bone plate to the bone. The image can be a craniocaudal radiograph used to determine the height of the fibular head relative to the joint surface and to select the screw lengths.

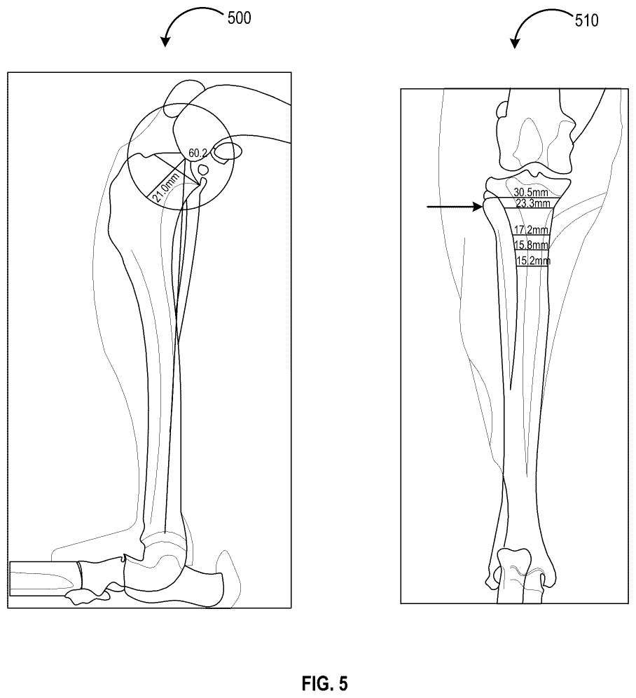

FIG. 5 illustrates basic imaging images in the form of a mediolateral radiograph 500 and craniocaudal radiograph 510 that can be used for pre-operative planning in accordance with specific embodiments of the inventions disclosed herein. Mediolateral radiographs can be captured using well known standard imaging techniques and so do not require specialized skills to obtain. The mediolateral radiograph 500 can be used to measure the TPA of the patient, which is 60.2 degrees, and to measure a desired radius of the arc of the osteotomy site, which in this case is 21 mm. These measurements are all that is required to select or define the operation of a surgical guide that will guide the osteotomy, guide the formation of the screw holes in the bone, and guide the translation of the bone to the appropriate rotation in accordance with specific embodiments of the inventions disclosed herein. The craniocaudal radiograph 510 can be used to measure desired screw lengths for the screws that will be placed in the bone plate. As illustrated, the screw lengths differ depending upon their location in the craniocaudal plane. These measurements are all that is required to select the length of the bone screws that will secure the bone plate to the bone in accordance with specific embodiments of the inventions disclosed herein. In specific embodiments of the invention, the three measurements taken from these two basic imaging images provide all the patient-specific information required for pre-operative planning of the TPLO.

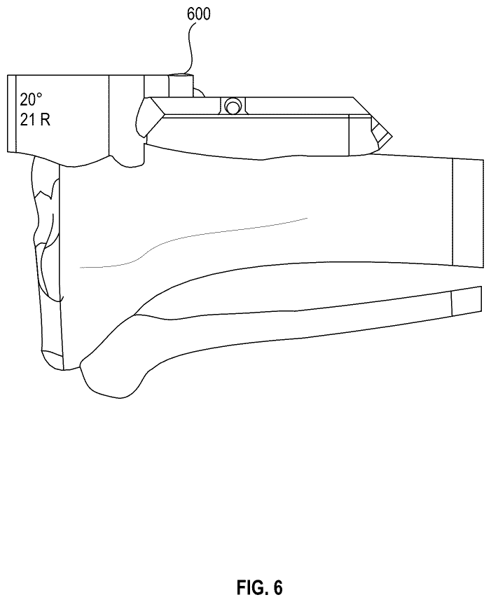

In specific embodiments of the invention, selection of the surgical guide from a library of surgical guides can be selected based on markings on the surgical guides what surgical conditions they are designed for. For example, the surgical guides could include markings which show they are designed for a specific TPA adjustment and a specific radius of the arc for the osteotomy. FIG. 6 illustrates a surgical guide 600 with an integrated drill guide and osteotomy guide that is labeled with markings for the surgical conditions it is designed for in accordance with specific embodiments of the inventions disclosed herein. As illustrated, surgical guide 600 is designed for a 20-degree TPA offset and a radius of 21 millimeters for the arc of the osteotomy guide. The implement has a bone facing surface that is curved. The implement includes a conforming bone facing surface that is shaped to conform to a standard tibial surface and was not generated for the specific tibia illustrated in the figures. However, given that the contour of a tibia is fairly standard across patients of a given size, the bone facing surface of the implement aligns well with the bone surface. The system has excellent fitment and accuracy due to the predictable surface contour of the proximal medial tibia of a patient of a given size. As such, although it appears the guide and other implements are created in a patient specific manner, they are created in a conformal manner.

In specific embodiments of the invention, the surgical guides are reusable because they are not patient specific. Furthermore, the surgical guides can include a blade facing surface and a bone facing surface that are made of a durable material such as metal. The surgical guides can therefore be reused for multiple surgeries and reduce the per-patient cost of the procedure in comparison to patient specific guides that are produced using customized additive manufacturing processes.

In specific embodiments of the invention, the surgical implements disclosed herein include conformal but not patient specific surgical implements. For example, the surgical guides and bone plates disclosed herein can include a conforming bone facing surface that is shaped to conform to a standard tibial surface while not being patient specific. The surgical guides can include portions that are conformal and other portions that are not. For example, the surgical guides could include portions that are conformal to a standard tibial surface close to the joint such as the proximal medial tibial cortex of the tibia's proximal diaphysis and metaphysis. The surface could include the osteotomy site as covered by the osteotomy guide 402 in FIG. 4 and the distal post-osteotomy bone segment as covered by the distal drill guide 403 in FIG. 4. In specific embodiments, the surgical guides could be conformal to a proximal medial tibial condyle, corresponding to the proximal pos-osteotomy bone as covered by the proximal drill guide 401 in FIG. 4. The conformal surface could be selected based on the anticipated variability of the anatomy encountered at the surface with low variability being favored for use as a surface for the surgical implements to be conformal to.

In specific embodiments of the invention, a conformal but not patient-specific surgical guide or bone plate can be part of a library of surgical guides from which a specific surgical guide is selected based on a measurement of the patient. For example, a dimensional profile of the proximal tibia can be measured, and a surgical guide or bone plate can be selected from the library based on that measurement where the surgical guides or bone plates in the library are designed to be conform to the tibia of patients having dimensional profiles in a given range. In specific embodiments, the measurement can alternatively be a tibial length which, when accompanied with an identification of a species of the patient, can be correlated with a specific tibial characteristic and corresponding entry in the library of implements. The guides in the library can include markings which indicate one or more of the sizes.

The drill guide and osteotomy guides can have bone facing surfaces which match the typical surface contour of the proximal medial canine tibia, for a patient of that size and conformation. The surgical guides can be developed using the systems described in U.S. Pat. App. No. 63/431,093 as filed on Dec. 8, 2022, which is incorporated by reference herein in its entirety for all purposes. The model can be developed using such systems and be based on average deviations in the three orthogonal planes observed across a patient population with similar size and conformation. Rather than depending on advanced imaging, such as CT imaging, to produce a patient specific 3D rendering for use in computer modeling software and 3D printing, the implements are generated based on average deviations from the three orthogonal planes in orthopedics (sagittal, frontal, and coronal) that the surface cortex of the proximal medial tibia displays, relative to those guidelines previously described for the ideal radial osteotomy, at specific anatomic locations across a population of size and breed matched controls. The surgical guides can then be generated and used to form a library of surgical guides with specific sizes. This provides a high degree of fitment when the guides are placed on the surface of the bone, while occupying the least amount of real estate on the tibial cortex and minimizing the 3D depth of the system necessary to fully constrain the effects of the intended guided actions. This preserves the surgeon's visualization of the proximal medial tibial cortex and increases the surgeon's confidence in the system guided actions. As used herein and with reference to cut guides, drill guides, bone plates, or integrated osteotomy guides and drill guides, the term “blade facing surface” will refer to the entire opposite side of the surgical implement to the bone facing surface (i.e., the side that faces away from the bone on which the implement is placed).

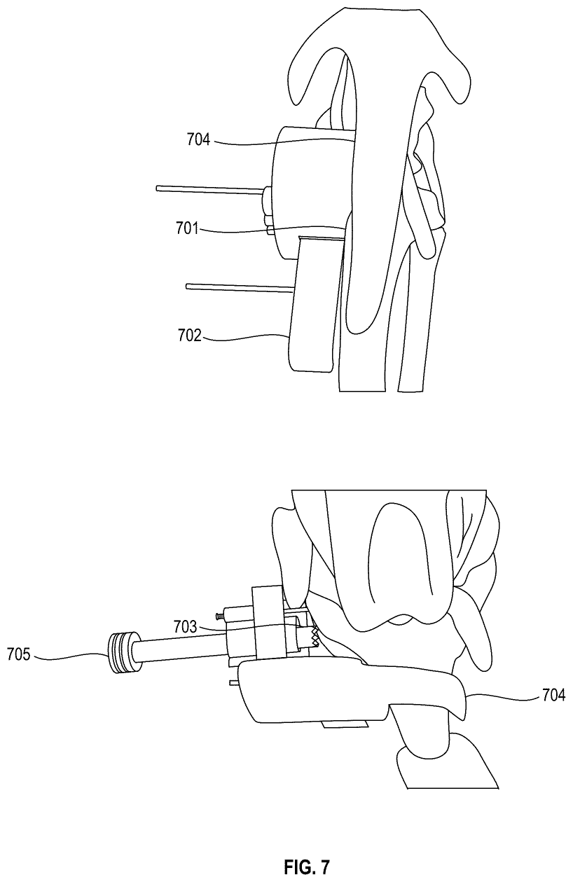

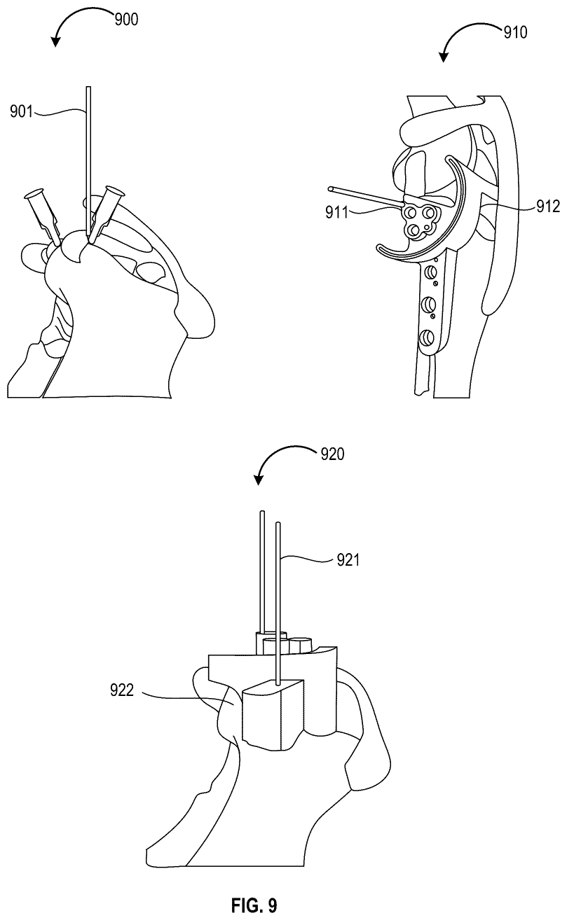

FIG. 7 illustrates the application of a conformal but not-patient specific surgical implement to the bone of a patient in accordance with specific embodiments of the inventions disclosed herein. FIG. 7 shows how the surgical implement includes a conformal portion that conforms to a surface of the tibia. FIG. 7 additionally shows how the surgical implement is not conformal along the entire bone facing surface of the surgical implement. For example, the distal drill guide is not flush with the tibia in a portion 702. Additionally, the surgical implement includes a deliberately non-conformal portion in the form of cut out 703 along a proximal drill guide which accounts for the predictable anatomic variability of the tibial condyle (due to soft tissue attachments at this location), which is the anatomic region of the tibia that the proximal drill guide is positioned upon. FIG. 7 also illustrates how a drill sleeve 705 can be inserted into a screw hole of a surgical guide to close the distance between a nonconformal surface of the surgical guide and the bone (e.g., the tibial condyle in the illustrated case). Additional non-conformal portions are possible in different embodiments or on the same surgical implement. For example, FIG. 9 illustrates a cut out 922 for the location of a standard popliteus muscle which has characteristics that vary significantly from patient to patient such that using a conformal, but not-patient specific surface might not produce a beneficial implement. FIG. 7 also illustrates how a surgical implement can include a patellar tendon shield 704 which extends away from a bone facing surface of the surgical implement and that protects the patellar tendon from the osteotomy by pushing it away from the planned cite of the osteotomy.

In specific embodiments of the invention the surgical guides disclosed herein can be accompanied by a positioner implement. The positioner implement can serve as a system positioner to assist a surgeon in position the surgical guides appropriately on a patient. The positioner implements, and other surgical implements disclosed herein, can include labels formed by etched or raised portions of the surface of the implement which labels their physical size or a physical size of a patient for which the implement should be applied.

The positioner implement can physically indicate a specific feature of the patient's tibia such as the intercondylar eminence. The physical indicator can be a mark on the positioner implement or be a result of the shape of the positioner implement (e.g., the positioner implement can be shaped to have a pointer and be configured to be positioned with the point of the pointer at the level of the intercondylar eminence).

The positioner implement can be configured as part of the surgical guide or can be configured to be detachable and connect to the surgical implement. The positioner implement can be positioned at a fixed orientation relative to the connected osteotomy guide and/or drill guide in such a way that if the physical indicator is placed relative to the portion of the tibia it is designed to indicate, then the surgical implement will be placed correctly. If the positioner is detachable, it can include an attachment adaptor to connect to a portion of the surgical guide. For example, it can include posts that connect with drill hole guides in the surgical guide. The system positioner can position the center point of the associated surgical guides directly, as opposed to traditional TPLO techniques which indirectly indicate the ideal center of the radial osteotomy.

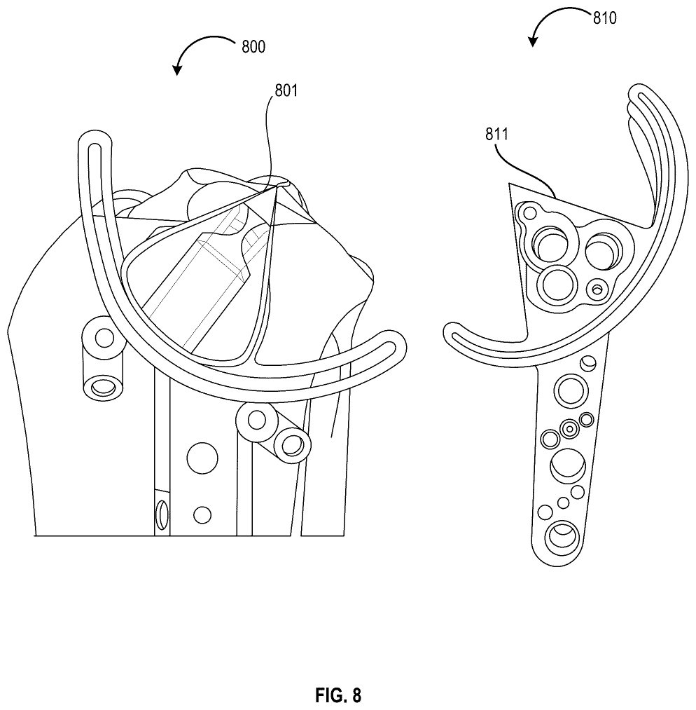

FIG. 8 illustrates positioner implements for surgical guides that are in accordance with specific embodiments of the inventions disclosed herein. In view 800, the surgical guide includes both an osteotomy guide and a drill guide, and positioner implement 801 is designed to be connected to the drill guide. The positioner implement 801 includes a marking which indicates that it is for a surgical condition requiring 21 mm of spacing between the intercondylar eminences and the osteotomy guide. The point of the positioner implement 801 can be accordingly placed by a surgeon at the level of the intercondylar eminences. Positioner implement 801 can be used with different surgical guides that each require a 21 mm spacing. The positioner implement can be selected from a library of positioner implements in which each entry of the library differs in terms of the spacing. In view 810, the surgical guide includes a positioner implement 811 that is part of a unibody element that defines the surgical guide. As with positioner implement 801, the point of the positioner implement 811 can be placed by a surgeon on the intercondylar eminences to appropriately distance the potential cite of the osteotomy from the joint of the patient. In accordance with specific embodiments of the invention, the level of the intercondylar eminences can be determined by performing a standard medial approach to the proximal tibia to delineate the joint surface with hypodermic needles.

In specific embodiments, the drill guides can include both pin guide and screw guides. The pin guides can be used to temporarily fix the drill guide or osteotomy guide in place while an articulating portion of a drill guide, the osteotomy guide, or an entire articulating drill guide, are moved into position. The pin guides can also be used to temporarily fix elements in place such an articulating element after it has been placed in its final position. Screw holes can then be formed in the bone using a drill while the pins maintain the elements in position.

FIG. 9 illustrates a process for placing a surgical implement on a patient's bone in using pins in accordance with specific embodiments of the inventions disclosed herein. View 900 shows the placement of a proximal alignment pin (PAP) 901. PAP 901 can be placed close to the joint surface such as 2-3 mm distal to the joint surface in the position of the medial collateral ligament. The PAP can be inserted according to the desired trajectory of the desired osteotomy. The trajectory of the PAP can then be accessed in the frontal plane and the coronal plane. The trajectory of the PAP can be adjusted by bending the pin at its junction with the tibial cortex until the desired trajectory is achieved. Accordingly, the pin can be relatively thin as compared to the bone screws that will be used to secure the bone plate to the patient bone in order to allow for bending of the pin.

View 910 shows how the surgical implement can be placed on the PAP 901. The illustrated surgical guide includes a pin hole 911 for the PAP 901 that is more proximate to the joint surface than any of the screw holes on the proximal drill guide. Accordingly, the screw holes can be formed with less concern for the screws impinging on the joint and causing damage thereto. As such, the screws do not need to be angled away from the joint as in prior art approaches and they can instead be perpendicular to the bone plate screw holes and therefore provide maximum structural support to the bone plate. View 910 also shows an embodiment with a patellar tendon shield 912 and how the patellar tendon shield 912 is positioned caudal to the patellar tendon.

View 920 shows how the surgical implement can be finally placed using a distal alignment pin (DAP) 921. As illustrated, the surgical guide can be rotated to center the distal screw guide on the bone, and the DAP 921 can then be placed through a dedicated pin hole on the surgical guide. Using this approach, the surgical guide can be precisely placed and oriented with minimal technical requirements placed on the surgeon besides placing the PAP proximate a commonly identified landmark and placing the DAP 921 in the center of the bone, which can be done by feeling for the front and back of the bone and placing the DAP 921 when the distal drill guide is half-way between the two. Accordingly, the surgical guide can be placed in a flexible manner with a conformal but not-patient specific bone facing surface to provide conformity where it is helpful and not where it is extraneous such that a library of surgical elements unacceptable large from a commercial perspective.

In specific embodiments of the invention after the drill guide has been properly placed on the patient bone, and potentially fixed in place using pins, the drill guide can be used to form screw holes in the patient bone. This process can be done using standard bone drills using screw holes on the drill guide as guides for fixing the drill in place. In specific embodiments, the process can be assisted by a drill sleeve that is placed through the screw holes of the drill guide while the bone is being drilled. The drill sleeve can be designed to be inserted into the screw holes of the drill guide without requiring excessive force on the drill guide, which may lead to unwanted modifications to the position of the drill guide relative to the patient's bone.

The screw hole of the drill guide may include protrusions in the screw hole that form a compression fit with the drill sleeve, but only at certain points along the length of the screw hole. The protrusions could include a set of inward facing protrusions around a bone facing surface edge of the screw hole and a second set of inward facing protrusions around a blade facing surface edge of the screw hole. The protrusions could be placed adjacent to a bone facing surface of the drill guide and a blade facing surface of the drill guide without and other protrusions in the screw hole. Accordingly, the drill sleeve can be held firmly in place to guide the drill while not requiring excessive force or friction to put the drill guide into place.

In specific embodiments, the osteotomy guide may be fully enclosed with precise tolerance as it relates to the osteotomy saw blade. This gives the osteotomy guide rigid control over the osteotomy starting position on the proximal medial tibial cortex and trajectory of the osteotomy through the tibia and center of osteotomy rotation when combined with the conforming fitment and methods for temporary stabilization such as pins inserted into the bone through placement holes on the osteotomy guide. Enclosing the osteotomy saw blade on more than two surfaces, with tight saw-blade-to-guide tolerance and an intimate interface between the osteotomy guide and tibial cortex, mitigates the risk for saw blade “skipping” along the sloped proximal medial tibial cortex or the surgeon altering the trajectory of the osteotomy in a non-desirable manner, as is observed with guide systems that afford unilateral support on either the concave or convex surface of the saw blade. These same risks exist for previously available guide systems enclosing greater than two surfaces of the saw blade as they have loose tolerance between the saw blade and guide wall, minimal or no conforming fitment at the tibial cortex to guide interface, and/or an excessive guide profile that obscures the surgeon's visualization as to where the guided actions are occurring relative to the tibial cortex. Various size osteotomy guides which correspond to an array of different size radial saw blades may be selected from a library of osteotomy guides having the characteristics of the osteotomy guides disclosed herein and organized based on an easy to acquire measurement of the patient such as the length of the patient's tibia. The osteotomy guides can also be selected based on any measurement related to the patient's proximal tibia in the region where the TPLO is to be performed.

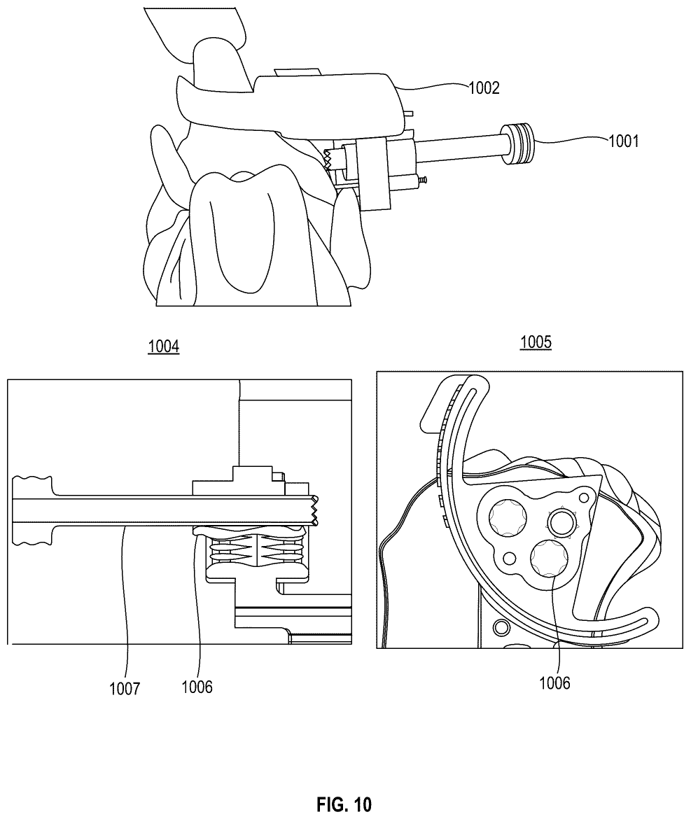

FIG. 10 illustrates a drill sleeve 1001 inserted into a screw hole of a drill guide of a surgical guide 1002 that is in accordance with specific embodiments of the inventions disclosed herein. As illustrated in cross sectional view 1004 and top-down view 1005, the drill guide screw holes can include protrusions to contact the drill sleeve. The protrusions can contact the drill sleeve in order to hold the drill in place when it is being applied to form a screw hole in the bone. However, the protrusions may not extend through the entire extend of the screw hole in the drill guide. For example, the protrusions can include a set of protrusions by the bone facing surface of the drill guide 1006 and a set of protrusions by the blade facing surface of the drill guide 1007. In accordance with these embodiments, inserting the drill sleeve into the drill guide may require less force, which would mean inserting the drill sleeve would be less likely to alter the position of the surgical guide. At the same time, the protrusions would still serve to keep the drill aligned when forming the screw hole in the bone.

In specific embodiments of the invention, once the osteotomy has been completed, the osteotomy guide can be removed and the bone plate that has been selected for use as the final stabilization of the post-osteotomy tibial bone segments can be applied using the screw holes that were previously formed in or marked on the bone segments prior to the osteotomy. The bone plate can be attached to the bone segments and the bone segments can be translated into alignment in various ways. The head of the plate can be secured to the proximal tibial bone segment with locking, or non-locking, bone screws placed via the pre-drilled or marked holes in the proximal bone segment. The body of the plate can be secured to the distal tibial bone segment with bone screws placed via the pre-drilled or marked holes in the distal bone segment.

In specific embodiments of the invention, the bone segments are brought into alignment using the bone plate, as attached to one of the bone segments, to apply leverage to the bone segment and bring the two bone segments into alignment. In keeping with these embodiments, a surgical implement in the form of a TPLO plate reduction tool which can be attached to the bone plate to provide a way to apply leverage to the bone plate. The surgical implement can include a nonrigid connector interface for a bone plate such as a hook that is configured to attach to a screw hole in the bone plate. The connector is nonrigid in that it can move with respect to at least one axis relative to the bone plate. The surgical implement can also include an interface for a lever tool. For example, the lever tool can be a Hohmann retractor and the interface for the lever tool can be a rectangular shaped receiver for the Hohmann as in the following diagram.

In specific embodiments of the invention, the surgical implements described in the prior paragraph can be used in a surgical method for a TPLO. The method can include attaching a bone plate to a first bone segment on a first side of the TPLO, attaching a surgical implement to the bone plate using a nonrigid connector interface on the surgical implement, attaching a lever tool to the surgical implement using an interface on the surgical implement, and translating the first bone segment using the lever tool. The method can also include engaging the lever tool with the caudal cortex of the tibia prior to translating the first bone segment. Engaging the lever tool with the caudal cortex provides a surface to push the lever tool against to provide a force for translating the bone segment.

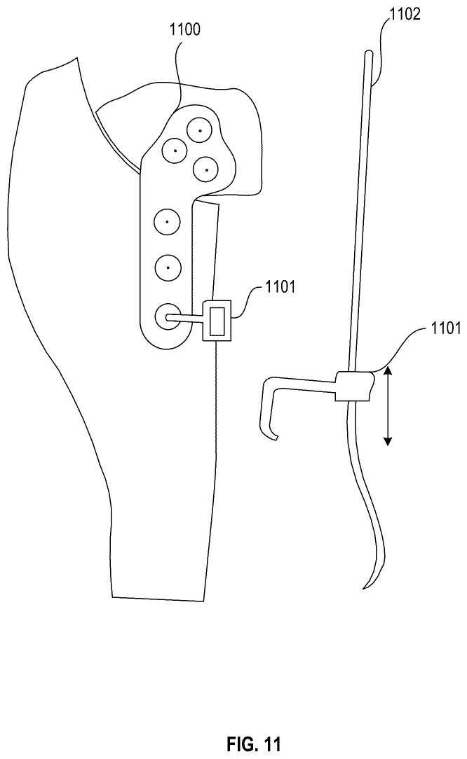

FIG. 11 illustrates a surgical implement for assisting with the translation of a patient bone using an attached bone plate in the form of a nonrigid connector interface and a lever tool in accordance with specific embodiments of the inventions disclosed herein. In FIG. 11, the nonrigid connector 1101 is a hook with an interface for a level tool. The lever tool in FIG. 11 is a Hohmann retractor 1102. As illustrated, the nonrigid connector 1101 can be connected temporarily to a screw hole of bone plate 1100, and then the Hohmann retractor 1102 can be inserted through the interface of the nonrigid connector 1101. The surgeon can then use Hohmann retractor 1102 to rotate the bone.

In specific embodiments of the approaches disclosed in the prior paragraphs, the tip of the Hohmann retractor is used to engage the caudal cortex of the tibia and functions as a lever to rotate the stem of the TPLO plate to align with the predrilled or marked holes for the corresponding bone screws, which are then placed. Once a bone screw has been placed, the TPLO plate reduction tool may be removed, and the final screw or screws can be placed.

In specific embodiments of the invention, another TPLO plate reduction tool that can be attached to the bone plate to provide a way to apply leverage to the bone plate can take the form of a rigid handle connected directly to the bone plate. The handle can include an interface for attaching to the bone plate and the bone plate can include a counter interface to attach the two. For example, the bone plate could include a separate set of screw holes to allow the handle to be screwed on to the bone plate. The handle could also include a surgeon handle portion for allowing the surgeon to grip the handle and rotate the bone.

In specific embodiments of the invention, the surgical implements described in the prior paragraph can be used in a surgical method for a TPLO. The method can include attaching a bone plate to a first bone segment on the first side of the TPLO, attaching a handle to the bone plate using an interface on the handle and the bone plate, and translating the first bone segment using the handle. The method can also include screwing the handle onto the bone plate.

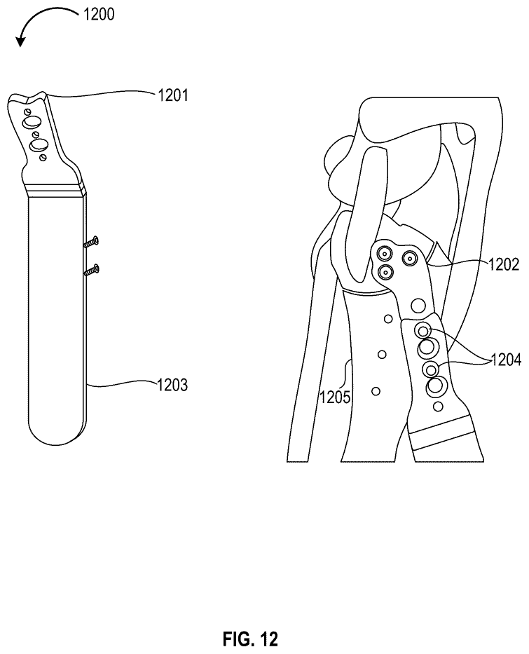

FIG. 12 illustrates a surgical implement for assisting with the translation of a patient bone using an attached bone plate in the form of an attachable handle in accordance with specific embodiments of the inventions disclosed herein. FIG. 12 includes handle 1200 having interface 1201 for attachment to bone plate 1202 and surgeon handle portion 1203. Handle 1200 can be screwed onto bone plate 1202 using screws as inserted into screw holes 1204 on bone plate 1202. As illustrated, the handle also includes gaps to allow bone screws to pass through the handle and fasten bone plate 1202 to screw holes in the patient bone 1205.

The utilization of a bone plate as a mechanism to rotate the tibial bone segments obviates the requirement for the rotation pin and stick pin. This in turn provides significant benefits in that it obviates risk for gap formation with poor stick pin placement, the risk of screw deflection from the stick pin, the risk of bone trauma/fracture from the rotation pin, and the risk of spatial void defects from the rotation pin, jig pin, and bone stress risers. This also reduces surgical assistant personnel requirement, which is a common requirement during stick pin placement, and reduces the number of technical steps to the procedure, leading to shorter surgery times.

In specific embodiments of the invention, a surgical method for a TPLO in accordance with the disclosure above is provided. The method includes contacting a tibia with an osteotomy guide, whereby the osteotomy guide defines a potential site for the TPLO. The method also includes marking a first side of the potential site with a first drill hole site using a drill guide that is attached to the osteotomy guide, marking a second side of the potential site with a second drill hole site using the drill guide, cutting the tibia at the potential site using the osteotomy guide, attaching a bone plate to the first side of the osteotomy site using the first drill hole site, translating a portion of the tibia using the bone plate so that the second drill hole site is aligned with a drill hole on the bone plate, and attaching the bone plate to the second side of the osteotomy site using the second drill hole.

The use of the bone plate to translate the bone segments into alignment is facilitated in part by other aspects of the surgical implements and procedures described below in that they maintain a parallel relationship amongst the trajectory of the radial osteotomy and the drill guides. This facilitates a fixed plane of rotation between the tibial bone segments and the ability to utilize the bone plate to achieve this rotation. Similarly, in certain approaches disclosed below, all screws of the bone plate are parallel which obviates the risk of inappropriate spatial offset between the bone plate and tibial cortex which can occur with non-parallel fixed angle locking screws.

In specific embodiments of the invention, the bone screws of the head of the bone plate and stem of the bone plate are all parallel to each other, and normal to the sagittal plane. This may result in a cranial/anterior trajectory of the bone screws in the bone plate stem relative to the surface/plane of the bone plate stem.

The drill guides and osteotomy guides disclosed herein may be designed to operate with an associated radial saw blade and bone plate, such that the bone “removed” by the radial saw blade is accounted for and compression across the osteotomy is achieved upon application of the bone plate. The system can be designed for the bone plate such that the holes drilled in the bone are offset to specifically account for the region of bone to be removed by the radial osteotomy blade, thus leading to compression across the entire osteotomy site once the bone plate is applied (without the need for eccentrically placed screws or reduction forceps as seen in the traditional TPLO system). This can be achieved, at least in part, by designing an offset into the screw holes of the bone plate and the drill guides. This can also be achieved, at least in part, by an offset introduced to the positioning of the drill guide relative to the osteotomy guide when the drill guide is attached. The offset can also bias the compression of the bone to assure solid contact on the trans-osteotomy side of the bone which can produced beneficial patient outcomes in that the portion of the bone which is furthest from the support of the bone plate is supported by the contact between bone segments.

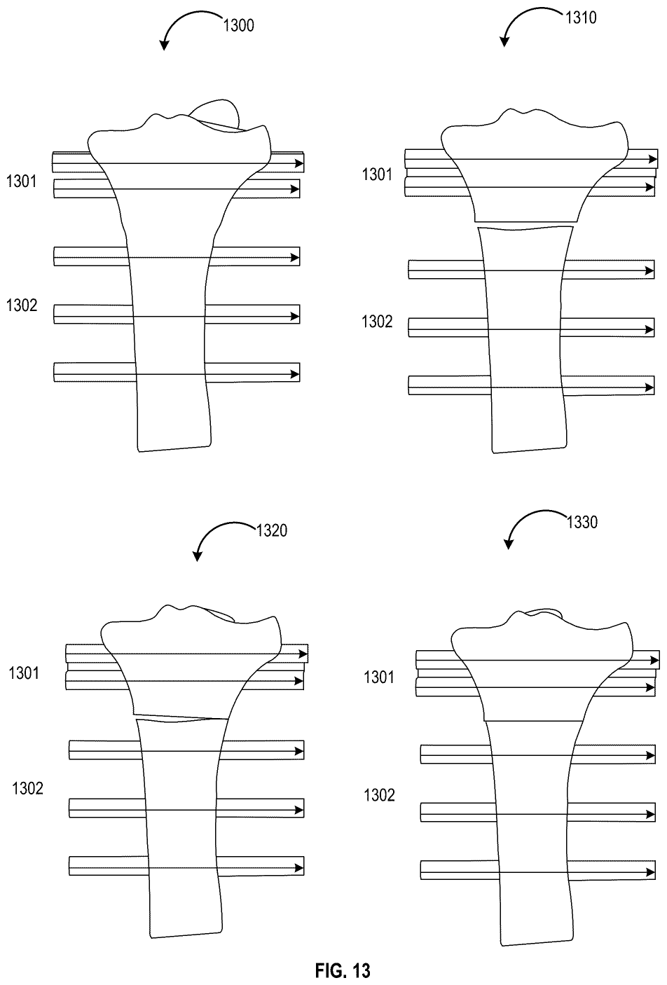

FIG. 13 illustrates screw trajectories for introducing a bias bone compression and a specific desired result of a biased bone compression in accordance with specific embodiments of the invention disclosed herein. Surgical implement (e.g., bone plate or bone screw) failure frequently occurs through cyclical fatigue such as through micro-bending of the implement over time. The risk of failure is greatest when the implant experiences cantilever bending due to a gap at the far side of the osteotomy (the trans-cortex). Therefore, while uniform osteotomy contact is ideal, trans-osteotomy bone to bone contact is essential.

FIG. 13 shows four views of a tibia as observed in the frontal plane from a caudal perspective to reveal the osteotomy more clearly. FIG. 13 illustrates the direction of screw holes that can be formed in a tibia using embodiments of the surgical implements disclosed herein in the form of cylinders traversing the tibia. The trajectories of the proximal drill holes are shown marked with arrows 1301, while the trajectories of the distal drill holes are shown with arrows 1302. For illustration purposes, the surgical implements used to form the screw holes and perform the osteotomy are not shown.

View 1300 shows the relative trajectory that the bone screws will assume once locked into a bone plate. As illustrated, the drill holes in the proximal segment are formed neutral to the proximal-distal (PD) axis via the drill guides, while the screw holes in the distal segment are formed with a two-degree distal orientation via the drill guides. In alternative embodiments, the two-degree offset created by these screw holes could instead by applied with the distal screw holes being formed neutral to the PD trajectory and the proximal screw holes being formed with a two-degree proximal orientation, or both sets of screw holes could have proximal or distal orientations. View 1310 shows the tibia after the radial osteotomy has been executed. No bone plate has been secured at this time; therefore, the distal pilot holes are still distally directed at two-degrees.

View 1320 shows the tibia after a bone plate (not shown) has been preliminarily secured, but the screws (not shown) have not yet been fully tightened. Engaging the bone screws to the bone plate normalizes the trajectory of the bone screws relative to the rigid bone plate. Therefore, the post-osteotomy tibial bone segments engaged by these bone screws experience an initial angular transformation. This leads to initial bone to bone osteotomy contact at the trans-osteotomy cortex. The trans-osteotomy cortex is the side opposite to where the bone plate would be secured when viewed from the frontal plane.

View 1330 shows the tibia after the bone screws are fully tightened and locked into the bone plate; the post osteotomy tibial bone segments experience a translational transformation. This reduces the distance between the proximal and distal post-osteotomy bone screws. This further compressing the osteotomy, encouraging uniform contact from the trans-cortex to the cis-cortex while ensuring trans-osteotomy contact. The cis-cortex is the bone surface beneath the bone plate. Accordingly, using offsets in the trajectory of the bone screws as defined by the drill guides and as defined by the bone plates can result in the desired degree of angular and translational movement to form the desired post-compression form of the bone.

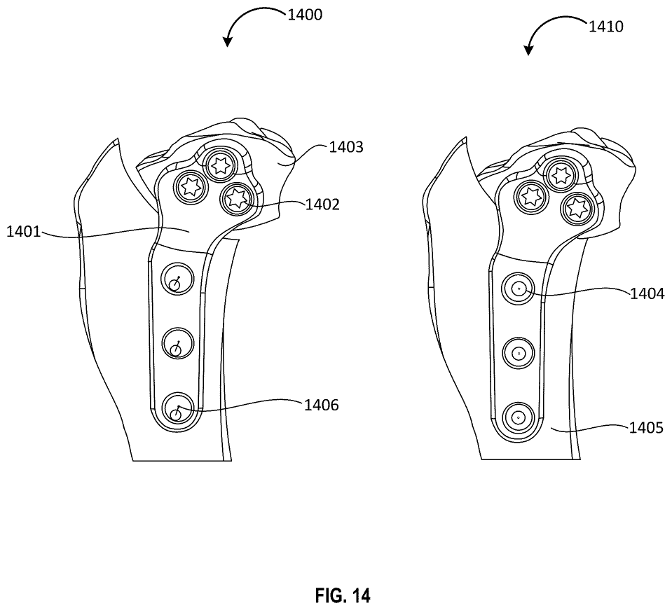

FIG. 14 illustrates a misalignment between a screw hole formed by a drill guide and a screw hole of a bone plate as used for introducing a bias bone compression in accordance with specific embodiments of the invention disclosed herein. In view 1400, the tibia is observed in the sagittal plane with a bone plate 1401 and bone screws 1402 secured to the proximal segment 1403. In view 1410, screw holes 1404 in distal segment 1405 have been subsequently aligned with screw holes in bone plate 1401. In view 1400, bone plate 1401 has been secured with three bone screws 1402 to one of the post-osteotomy tibial bone segments (proximal segment 1403 depicted in this example), which has been rotated but not compressed. Relative to the final position that the bone screws will assume, centered within their associated locking bone screw hole in the bone plate, the screw holes within the remaining post-osteotomy tibial bone segment (distal segment 1405 depicted here) are eccentrically drilled. The direction and magnitude of this eccentricity is determined by the direction and magnitude of desired translational osteotomy compression shown be lines 1406. As bone screws are fully tightened and locked into bone plate 1401 and the remaining post-osteotomy tibial bone segment (distal segment 1405 depicted here), the post osteotomy tibial bone segments experience a translational transformation (reducing the distance between the proximal and distal post-osteotomy bone screws). This compresses the osteotomy uniformly along its radial surfaces.

In specific embodiments, both the trajectory of the screw holes and the location of the screw holes can be set, as illustrated in FIGS. 13 and 14 in order to form the desired compression in the bone segments in order to account for the kerf of the blade that forms the osteotomy and to form the desired trans-osteotomy contact required to stabilize the bone.

In specific embodiments of the invention, a surgical method for a TPLO in accordance with the disclosure above is provided. The method includes contacting a tibia with a surgical implement, wherein the surgical implement comprises: (i) a bone facing surface; (ii) a first set of screws holes on a distal side of the TPLO; (iii) and a second set of screw holes on a proximal side of the TPLO. The method also includes attaching the surgical implement to the distal side of the TPLO with a first set of screws inserted into the first set of screw holes, wherein the first set of screw holes are aligned relative to the bone facing surface so that the first set of screws are inserted substantially parallel to each other. The method also includes attaching the surgical implement to the proximal side of the TPLO with a second set of screws inserted into the second set of screw holes, wherein the second set of screw holes are aligned relative to the bone facing surface so that the second set of screws are inserted substantially parallel to each other. The surgical implement can be one of a surgical guide and a bone plate.

In specific embodiments of the invention, the surgical guides are adjustable. The surgical guides can include a conforming bone facing surface that conforms to a standard tibial surface and is not patient specific and can also include a set of at least two adjustment entities (e.g., screws, pins with set screws, etc.). Screws can be threaded through nuts that are fixed to the body of the surgical guide. The body can include the conforming bone facing surface and the blade facing surface of the surgical guide. The entities can be in contact with the surface of the tibia to allow adjustments of the alignment of the conforming bone facing surface and blade facing surface relative to the tibia. The set of entities used for adjustment can include at least three entities to provide for adjustment. One entity (screw, pin with set screw, or similar) can be sufficient to alter the trajectory of the guides in one axis (proximodistal or craniocaudal). Two entities, if placed functionally orthogonal, can be sufficient to alter system trajectory in both the proximodistal and craniocaudal axes. Two screws may be placed functionally in the same axis but on opposing “sides” of the system to alter the system trajectory in that axis in a positive or negative capacity. In specific embodiments, the system has three screws. The system may need to be altered in the craniocaudal axis in either a cranial or caudal angulation, thus there are two entities; however, the system trajectory in the proximodistal axis most frequently would need augmentation to angle the system trajectory more distally, away from the joint surface, thus there is only one entity in this axis.

The guides may include temporary stabilization wires. These provide further stabilization of the system on the tibial cortex and visually display the trajectory of the guidance system, as these wire guides have the same trajectory as the guidance system. These temporary stabilization wires may be positioned within the guidance system with specific relation to identifiable anatomic landmarks, such that their precise location, and therefore position of the guidance system relative to the bone surface, can be planned pre-operatively and confidently executed intra-operatively. When there is slight variability in the surface contour of the patient's proximal medial tibial cortex, the surgeon may utilize one of various methods to precisely modify the trajectory of the system. For example, threaded offset posts may be present to create a magnitude of offset between the guidance system and tibial cortex, thereby tilting the guidance system and modifying its trajectory.

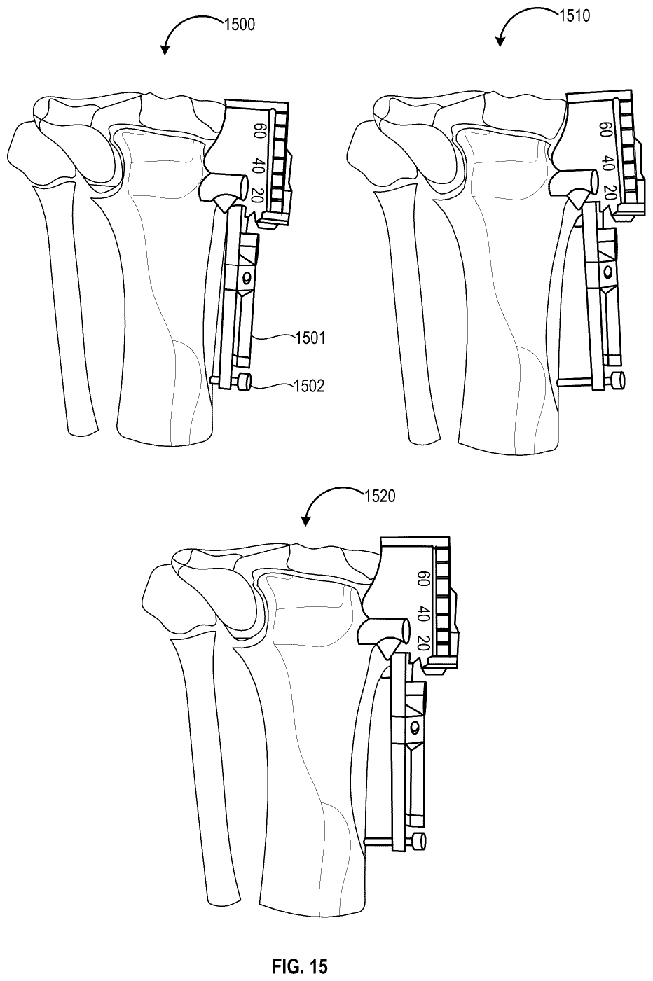

FIG. 15 illustrates the three views of a surgical guide with adjustment screws to modify the trajectory of a drill guide and osteotomy guide on the surgical guide in accordance with specific embodiments of the inventions disclosed herein. Examples of the magnitude of the modified trajectory can be seen in the view of FIG. 15. Set screws may be utilized with wires or pins in a similar manner FIG. 15 shows a surgical guide 1501 with adjustment screw 1502. In the view 1500, the drill guide has a sagittal plane trajectory offset screw at the distal end of the surgical guide. In view 1500, this screw has been loosened thus reducing the distance from the distal end of the surgical guide to the bone. This sets the trajectory of the surgical guide, such that any osteotomy, screw holes, or pins that are applied to the bone using the surgical guide are directed more proximally (i.e., the osteotomy exits the lateral cortex of the tibia closer to the joint than it started). In view 1510, the screw has been tightened, thus increasing the distance from the distal end of the surgical guide to the bone. This sets the trajectory of the surgical guide, such that any osteotomy, screw holes, or pins that are applied to the bone using the surgical guide are directed more distally (i.e., the osteotomy exits the lateral cortex of the tibia further from the joint than it started). In view 1520, the trajectory offset screw at the distal end of the surgical guide is in the neutral position. Using the illustrated approach, a surgical guide can include a conformal bone facing surface of a surgical guide that is non-patient specific while still allowing a surgeon to precisely apply a blade, screw hole, or pin to the tibia in a desired location and with a desired trajectory.

In specific embodiments of the invention, the surgical implements disclosed herein can be configured to be aligned relative to the tibia to provide certain angles relative to the sagittal, coronal, or transverse planes for the trajectory of blades, screws, or pins that will be applied to the tibia using the surgical implements. The alignment can be set such that a set of drill guide holes on a surgical implement are aligned relative to the bone facing surface such that screws inserted into the set of drill guide holes are perpendicular to the sagittal plane while the set of drill guide holes are off-normal from a blade facing surface of the implement. The surgical implement in this example can be a surgical drill guide or a bone plate. There are numerous benefits to this approach in that the rotation and alignment of the two bone segments is simplified to a single axis of rotation from the pre-osteotomy to the final fixated position of the bone segments. Furthermore, the parallel screws increase the fixing strength of the bone plate. Using the approaches disclosed herein parallel screw insertion into the proximal tibial bone segment is possible due to the increased control and confidence afforded by the drill guides and associated procedures which assure that the screws will not be directed into the joint and because there are no longer pin holes to avoid as in prior systems. The surgical implements can be configured to provide for this type of alignment in various ways. For example, the surgical implements can be adjustable such as via adjustment screws. As another example, the surgical implements can have conformal surfaces that are patient specific. As another example, the surgical implements can have conformal surfaces that are not patient specific and that are adjustable.

In specific embodiments of the invention, a surgical implement for a TPLO is provided. The implement includes a bone facing surface, a first set of screws holes on a distal side of the TPLO, and a second set of screw holes on a proximal side of the TPLO. The first set of screw holes can be aligned relative to the bone facing surface so that screws inserted into the first set of screw holes are substantially parallel to each other. The second set of screw holes can be aligned relative to the bone facing surface so that screws inserted into the second set of screw holes are substantially parallel to each other.

In specific embodiments of the invention, the bone plates can have offsets in terms of the angle formed by the head of the bone plate relative to the stem. In specific embodiments, the head has an offset relative to the stem of the bone plate that is rotational in character and centered on a rotational pivot point that is specific to its intended radial osteotomy blade. These bone plates can be in a library of surgical implements where the entries in the library differ in terms of the offset angle. The bone plate offsets can provide a surgeon with the ability to alter a patient's final TPA, even after pre-drilling the screw holes. These bone plates can be referred to as TPA offset bone plates, which have a deliberate rotational offset of their locking screws within the bone plate head, so that the surgeon may increase or decrease the final TPA by a predetermined finite amount. This is possible as these plates are designed with different locking screw orientations in the bone plate head, such that application of the TPA offset bone plate will increase or decrease the magnitude, degree, or chord, rotation of the tibial bone segments, using the same predrilled screw holes. These bone plates offsets can also be designed to work with a drill guide that is designed to be placed at specific fixed angles relative to a neutral position such as the drill guides that are designed to move between lock points as described above. In combination with such an approach, a library of TPA offset bone plates can allow a surgeon to access more potential angles of adjustment for the TPA than the drill guide alone would allow with its fixed angles set by the available lock points.

In specific embodiments of the invention, a library of surgical implements for a TPLO is provided. The library comprises a bone plate, a set of negative angle offset bone plates wherein each negative angle offset bone plate in the set of negative angle offsets has a head with a negative angle relative to the bone plate that is unique amongst the negative angle offset bone plates in the set of negative angle offset bone plates, and a set of positive angle offset bone plates wherein each positive angle offset bone plate in the set of positive angle offsets has a head that has a positive angle relative to the bone plate and that is unique amongst the positive angle offset bone plates in the set of positive angle offset bone plates. The bone plates can be labeled with their offsets using etched or raised material or other markings. The markings can distinguish the bone plates in a library of bone plates. The bone plates can also be labeled with the radial blade size they are intended to be used with.

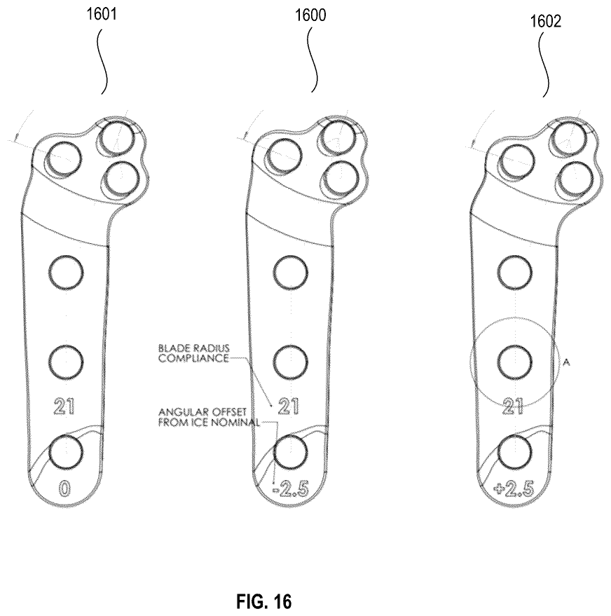

FIG. 16 illustrates offset plates that can be used in accordance with specific embodiments of the inventions disclosed herein. In the illustrated example, a bone plate 1601 is shown with a positive offset plate 1602 and a negative offset plate 1600. Bone plate 1601 with no offset can be referred to as the neutral bone plate. The bone plates will result in the magnitude of TPA rotation related to the guidance system descriptors which in this case are etched markings. The negative offset plate 1600 is marked “−2.5” degrees. This plate will reduce the magnitude of rotation by 2.5 degrees. The positive offset plate 1602 is marked “+2.5.” Each of the plates is marked to indicate the radial blade size “21” the bone plate is intended to operate with where “21” indicates a 21 mm radial TPLO blade.

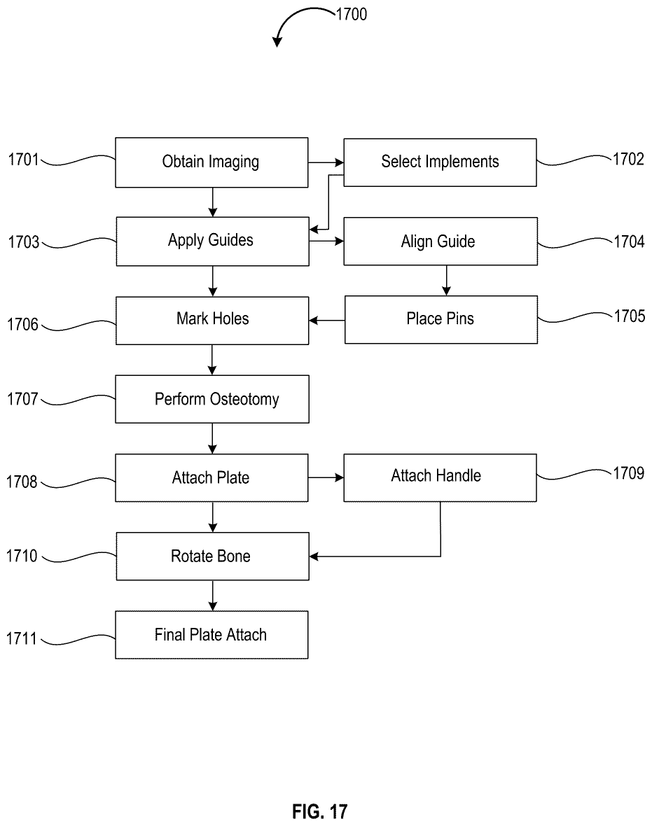

FIG. 17 illustrates a flow chart 1700 of a set of surgical methods for a TPLO that are in accordance with embodiments of the inventions disclosed herein. The methods of flow chart 1700 include a step 1701 of obtaining an image of a patient bone to determine a patient tibial plateau angle. The image can be a mediolateral radiograph. Flow chart 1700 also includes a step 1703 of applying the surgical guide to the patient bone. Flow chart 1700 also includes a step 1707 of performing a tibial plateau leveling osteotomy of the patient bone using the surgical guide.

Flow chart 1700 also includes an optional step 1702 of selecting at least one of a surgical guide and a bone plate from a library based on the patient tibial plateau angle. The library can be a library of bone plates which includes the bone plate. The bone plate can be complementary to the surgical guide in the library of bone plates. The surgical guide can include a conformal bone facing surface, and the conformal bone facing surface can conform to a standard tibial surface and is not patient specific. The bone plate can be selected from a library in step 1702 and step 1702 can further comprise selecting an offset bone plate to be used after performing the tibial plateau leveling osteotomy. The offset plate can be selected from: (i) a set of at least one negative angle offset bone plates wherein each negative angle offset bone plate in the set of negative angle offsets has a head with a negative angle relative to the bone plate; and (ii) a set of at least one positive angle offset bone plates wherein each positive angle offset bone plate in the set of positive angle offsets has a head that has a positive angle relative to the bone plate.

In specific embodiments, step 1701 can include obtaining a craniocaudal radiograph image of the patient bone and step 1702 can include selecting lengths for the set of bone screws using the craniocaudal radiograph. The lengths can then be used to select screws from a library of surgical implements to perform the surgery.

Flow chart 1700 also includes a step 1706 of marking screw holes on the patient bone. This step can involve drilling a proximal screw hole and a distal screw hole in the patient bone using a drill guide of the surgical guide. The screw holes can be pilot holes for future bone screws. The step can alternatively involve making an indication on the bone to show where a future screw hole should be formed. In specific embodiments, the marking (e.g., drilling) can be conducted prior to the performing of the tibial plateau leveling osteotomy as in step 1707.

Flow chart 1700 also includes a step 1708 of attaching a bone plate to the patient bone using: (i) a distal bone screw in a distal bone plate screw hole that is aligned with the distal screw hole; and (ii) a proximal bone screw in a proximal bone plate screw hole that is aligned with the proximal screw hole. Flow chart 1700 also includes a step 1710 of rotating the patient bone. The rotating can be done using the bone plate as a handle after the bone plate is attached to the bone. The drill guide and the bone plate can be complementary in that: (i) the distal guide screw hole and the proximal guide screw hole are aligned with a planned pre-operative orientation of a planned distal bone screw location and a planned proximal bone screw location; and (ii) the distal bone plate screw hole and the proximal bone plate screw hole are aligned with a planned post-operative orientation of the planned distal bone screw location and the planned proximal bone screw location. Flow chart 1700 concludes with a step 1711 of conducting a final attach of the bone plate to the bone by attaching bone plate to the side of the osteotomy that it has not previously been attached to.

Flow chart 1700 also includes optional steps including a step 1704 of aligning the guide and a step 1705 of placing pins on the bone through the guide. Step 1704 could comprise aligning a positioner implement with the intercondylar eminences of the patient bone while applying the surgical guide to the patient bone. The surgical guide could include an osteotomy guide and a proximal drill guide. The osteotomy guide could form an arc for the tibial plateau leveling osteotomy. The proximal drill guide could include a positioner implement. The positioner implement could extend towards the center of the arc. The positioner implement could be more proximal than any screw hole in the proximal drill guide. Accordingly, when the guide is aligned the surgeon could be assured that the screw holes are sufficiently distal to the joint of the patient.

The steps can be conducted iteratively as additional pins are placed. For example, the guide can be aligned, a first pin can be placed to assist in a second alignment, and then a second pin can be placed. The first pin could be a PAP. The second pin could be a DAP. Step 1705 could comprise placing a proximal alignment pin in a proximal alignment pin hole in a proximal drill guide of the surgical guide after applying the surgical guide to the patient bone. The proximal alignment pin hole could be more proximal than any screw hole in the proximal drill guide. Step 1704 could then be conducted in a second iteration and involve aligning a distal alignment pin hole in distal drill guide of the surgical guide on a center of the patient bone after placing the proximal alignment pin. Step 1705 could then be conducted in a second iteration and involve placing a distal alignment pin in the distal alignment pin hole after aligning the distal alignment pin hole. After such a process, step 1706 could involve drilling a proximal screw hole and a distal screw hole in the patient bone using the drill guide after placing the distal alignment pin.

Flow chart 1700 also includes a step 1708 of attaching a bone plate to the patient bone using a set of bone screws. Flow chart 1700 also includes a step 1709 of attaching a rotation handle to the bone plate. The bone plate can have a rotation handle interface to fix the bone plate to the rotation handle in combination with the bone plate interface. Step 1710 of flow chart 1700 can include rotating, after performing the tibial plateau leveling osteotomy, the patient bone using the rotation handle.

In specific embodiments of the invention, a surgical guide for a TPLO is provided. The guide comprises an osteotomy guide defining a potential site for the TPLO and a unibody drill guide connected to the osteotomy guide and spanning the potential site for the tibial plateau leveling osteotomy. In specific embodiments of the invention, a surgical guide for a TPLO is provided. The guide comprises an osteotomy guide defining a potential site for the TPLO and a drill guide having a proximal drill guide and a distal drill guide. The guide further comprises a unibody element spanning the potential site for the tibial plateau leveling osteotomy. The unibody element includes the osteotomy guide and one of: (i) the proximal drill guide; and (ii) the distal drill guide.

In specific embodiments of the invention, a surgical method for a TPLO is provided. The method comprises contacting a tibia with an osteotomy guide, whereby the osteotomy guide defines a potential site for the TPLO, marking a first side of the potential site for a first drill hole, marking a second side of the potential site for a second drill hole, wherein the marking for the first drill hole and the marking for the second drill hole are done to align a proximal portion of the tibia and a distal portion of the tibia via a bone plate attached using a first screw through the first drill hole and a second screw through the second drill hole, cutting the tibia at the potential site, selecting a second bone plate from a library of bone plates, wherein the second bone plate and each bone plate in the library of bone plates has an angle offset from the bone plate, aligning a proximal portion of the tibia and a distal portion of the tibia so that the first drill hole and the second drill hole are aligned with a pair of drill holes on the second bone plate, and attaching the second bone plate using the first screw inserted into the first drill hole and the second screw inserted into the second drill hole.

In specific embodiments of the invention, a surgical method for a TPLO is provided. The method comprises contacting a tibia with an osteotomy guide, whereby the osteotomy guide defines a potential site for the TPLO. The method also comprises marking a first side of the potential site for a first drill hole, marking a second side of the potential site for a second drill hole, wherein the marking for at least one of the first drill hole and second drill hole is done using a drill guide attached to the osteotomy guide and configured to be adjustable between a set of lock points relative to the osteotomy guide, cutting the tibia at the potential site using the osteotomy guide, and selecting a bone plate from a library of bone plates, wherein each bone plate in the library of bone plates has a unique angle offset. The method also comprises aligning a proximal portion of the tibia and a distal portion of the tibia so that the first drill hole and the second drill hole are aligned with a pair of drill holes on the bone plate and attaching the bone plate using a first screw inserted into the first drill hole and a second screw inserted into the second drill hole.

In specific embodiments of the invention, a surgical guide for a tibial plateau leveling osteotomy is provided. The surgical guide comprises a blade facing surface with an osteotomy guide for the tibial plateau leveling osteotomy. The surgical guide comprises a conforming bone facing surface. The surgical guide comprises a set of at least two adjustment screws. The conforming bone facing surface conforms to a standard tibial surface and is not patient specific. The set of at least two adjustment screws are positioned to adjust an alignment of the conforming bone facing surface.

In specific embodiments of the invention a surgical method for a tibial plateau leveling osteotomy is provided. The method comprises measuring a patient. The method comprises selecting a surgical guide for the tibial plateau leveling osteotomy from a library of surgical guides based on the measuring. The library of surgical guides includes a set of surgical guides, and each surgical guide in the set of surgical guides comprises: (i) a blade facing surface with an osteotomy guide; and (ii) a conforming bone facing surface which conforms to a standard tibial surface for patients having a measurement in a unique range of measurements for the surgical guide amongst the surgical guides in set of surgical guides.

In specific embodiments of the invention, a surgical guide for a tibial plateau leveling osteotomy is provided. The surgical guide comprises an osteotomy guide forming an arc for the tibial plateau leveling osteotomy. The surgical guide comprises a drill guide attached to the osteotomy guide and configured to rotate along the arc. In specific embodiments, the surgical guide comprises a proximal drill guide of the surgical guide fixed relative to the osteotomy guide. The drill guide is a distal drill guide. In specific embodiments, the surgical guide further comprises a distal drill guide of the surgical guide fixed relative to the osteotomy guide. The drill guide is a proximal drill guide. The drill guide spans the osteotomy guide. The drill guide includes a distal drill guide and a proximal drill guide.

In specific embodiments, a surgical guide for a tibial plateau leveling osteotomy is provided. The surgical guide comprises an osteotomy guide forming an arc for the tibial plateau leveling osteotomy. The surgical guide comprises a set of markings on the osteotomy guide where the markings indicate a set of offset angles along the arc for a drill guide that attaches to the osteotomy guide.

In specific embodiments, a surgical guide for a tibial plateau leveling osteotomy is provided. The surgical guide comprises an osteotomy guide defining a potential site for the tibial plateau leveling osteotomy. The surgical guide comprises a drill guide having a proximal drill guide and a distal drill guide. The surgical guide comprises a unibody element spanning the potential site for the tibial plateau leveling osteotomy. The unibody element includes the osteotomy guide and at least one of: (i) the proximal drill guide; and (ii) the distal drill guide.

In specific embodiments, a surgical guide for a tibial plateau leveling osteotomy is provided. The surgical guide comprises an osteotomy guide defining a potential site for the tibial plateau leveling osteotomy. The surgical guide comprises a unibody drill guide connected to the osteotomy guide and spanning the potential site for the tibial plateau leveling osteotomy.

In specific embodiments, a method for a TPLO is provided. The method comprises contacting a tibia with an osteotomy guide, whereby the osteotomy guide defines a potential site for the tibial plateau leveling osteotomy. The method comprises marking a first side of the potential site for a first drill hole using a drill guide that is attached to the osteotomy guide. The method comprises rotating the drill guide along an arc formed by the osteotomy guide. The method comprises marking, after rotating the drill guide, a second side of the potential site for a second drill hole using the drill guide. The method comprises cutting the tibia at the potential site using the osteotomy guide. The method comprises aligning a proximal portion of the tibia and a distal portion of the tibia so that the first drill hole and the second drill hole are aligned with a pair of drill holes on a bone plate. The method comprises attaching the bone plate using a first screw inserted into the first drill hole and a second screw inserted into the second drill hole.

In specific embodiments, a surgical method for a tibial plateau leveling osteotomy is provided. The method comprises contacting a tibia with an osteotomy guide, whereby the osteotomy guide defines a potential site for the tibial plateau leveling osteotomy. The method comprises marking a first side of the potential site with a first drill hole site using a drill guide that is attached to the osteotomy guide. The method comprises marking a second side of the potential site with a second drill hole site using the drill guide. The method comprises cutting the tibia at the potential site using the osteotomy guide. The method comprises attaching a bone plate to the first side of the osteotomy site using the first drill hole. The method comprises translating a portion of the tibia, using the bone plate, so that the second drill hole is aligned with a drill hole on the bone plate. The method comprises attaching the bone plate to the second side of the osteotomy site using the second drill hole.

In specific embodiments, a surgical method for a tibial plateau leveling osteotomy is provided. The method comprises contacting a tibia with a surgical implement, wherein the surgical implement comprises: (i) a bone facing surface; (ii) a first set of screws holes on a distal side of the tibial plateau leveling osteotomy; (iii) and a second set of screw holes on a proximal side of the tibial plateau leveling osteotomy. The method comprises attaching the surgical implement to the distal side of the tibial plateau leveling osteotomy with a first set of screws inserted into the first set of screw holes, wherein the first set of screw holes are aligned relative to the bone facing surface so that the first set of screws are inserted substantially parallel to each other. The method comprises attaching the surgical implement to the proximal side of the tibial plateau leveling osteotomy with a second set of screws inserted into the second set of screw holes, wherein the second set of screw holes are aligned relative to the bone facing surface so that the second set of screws are inserted substantially parallel to each other. The surgical implement is one of a surgical guide and a bone plate.

In specific embodiments, a surgical implement for a tibial plateau leveling osteotomy is provided. The surgical implement comprises a bone facing surface. The surgical implement comprises a first set of screws holes on a distal side of the tibial plateau leveling osteotomy. The surgical implement comprises a second set of screw holes on a proximal side of the tibial plateau leveling osteotomy. The first set of screw holes are aligned relative to the bone facing surface so that screws inserted into the first set of screw holes are substantially parallel to each other. The second set of screw holes are aligned relative to the bone facing surface so that screws inserted into the second set of screw holes are substantially parallel to each other.

In specific embodiments of the invention, a surgical implement for a tibial plateau leveling osteotomy is provided. The surgical implement comprises a nonrigid connector interface for a bone plate. The surgical implement also comprises an interface for a lever tool.

In specific embodiments of the invention, a surgical method for a tibial plateau leveling osteotomy is provided. The method comprises attaching a bone plate to a first side of the tibial plateau leveling osteotomy. The method comprises attaching a surgical implement to the bone plate using a nonrigid connector interface on the surgical implement. The method comprises attaching a lever tool to the surgical implement using an interface on the surgical implement. The method comprises translating the first side of the tibial plateau leveling osteotomy using the lever tool. The method comprises engaging the lever tool with the caudal cortex of the tibia prior to translating the first side of the tibial plateau.

In specific embodiments of the invention, a surgical guide for a tibial plateau leveling osteotomy is provided. The surgical guide forms an arc for the tibial plateau leveling osteotomy. The surgical guide comprises a drill guide attached to the osteotomy guide and configured to be adjustable between a set of lock points relative to the osteotomy guide.

In specific embodiments of the invention, a library of surgical implements for a tibial plateau leveling osteotomy is provided. The library comprises a bone plate. The library comprises a set of negative angle offset bone plates wherein each negative angle offset bone plate in the set of negative angle offsets has a head with a negative angle relative to the bone plate that is unique amongst the negative angle offset bone plates in the set of negative angle offset bone plates. The library comprises a set of positive angle offset bone plates wherein each positive angle offset bone plate in the set of positive angle offsets has a head that has a positive angle relative to the bone plate and that is unique amongst the positive angle offset bone plates in the set of positive angle offset bone plates.

In specific embodiments of the invention, a surgical method for a tibial plateau leveling osteotomy is provided. The method comprises contacting a tibia with an osteotomy guide, whereby the osteotomy guide defines a potential site for the tibial plateau leveling osteotomy. The method comprises marking a first side of the potential site for a first drill hole. The method comprises marking a second side of the potential site for a second drill hole, wherein the marking for the first drill hole and the marking for the second drill hole are done to align a proximal portion of the tibia and a distal portion of the tibia via a bone plate attached using a first screw through the first drill hole and a second screw through the second drill hole. The method comprises cutting the tibia at the potential site. The method comprises selecting a second bone plate from a library of bone plates, wherein the second bone and each bone plate in the library of bone plates has an angle offset from the bone plate. The method comprises aligning a proximal portion of the tibia and a distal portion of the tibia so that the first drill hole and the second drill hole are aligned with a pair of drill holes on the second bone plate. The method comprises attaching the second bone plate using the first screw inserted into the first drill hole and the second screw inserted into the second drill hole.

In specific embodiments of the invention, a surgical method for a tibial plateau leveling osteotomy is provided. The method comprises contacting a tibia with an osteotomy guide. The osteotomy guide defines a potential site for the tibial plateau leveling osteotomy. The method comprises marking a first side of the potential site for a first drill hole. The method comprises marking a second side of the potential site for a second drill hole, wherein the marking for at least one of the first drill hole and second drill hole is done using a drill guide attached to the osteotomy guide and configured to be adjustable between a set of lock points relative to the osteotomy guide. The method comprises cutting the tibia at the potential site using the osteotomy guide. The method comprises selecting a bone plate from a library of bone plates, wherein each bone plate in the library of bone plates has a unique angle offset. The method comprises aligning a proximal portion of the tibia and a distal portion of the tibia so that the first drill hole and the second drill hole are aligned with a pair of drill holes on the bone plate. The method comprises attaching the bone plate using a first screw inserted into the first drill hole and a second screw inserted into the second drill hole.

In specific embodiments of the invention, a set of tibial plateau leveling surgical implements is provided. The set of tibial plateau leveling surgical implements comprises a surgical guide having a blade facing surface and a conforming bone facing surface, wherein the conforming bone facing surface includes drill hole guides. The set of tibial plateau leveling surgical implements comprises a detachable positioner implement that physically indicates a location and that includes an adaptor for connecting to the surgical guide in a fixed orientation.

As stated in the summary above, surgical procedures associated with specific embodiments of the surgical implements disclosed herein, can be conducted with traditional imaging. However, in alternative embodiments, proprietary modeling tools such as those disclosed in U.S. Pat. App. No. 63/431,093, filed Dec. 8, 2022, and incorporated by reference herein in its entirety for all purposes, may be utilized to generate a predictive three-dimensional (3D) rendering of the patient's tibia, based on anatomic measurements obtained from that patient's planar radiograph, for planning with a 3D guidance system template. However, specific embodiments of the inventions disclosed herein can be conducted solely with two-dimensional imaging and standard pre-operative planning.

In specific embodiments disclosed herein, the surgical implements are functionally modular with respect to which actions are guided by the surgical implements. For example, a surgeon may elect to incorporate either a radial saw guide or a drill guide individually or utilize both in combination. The implements disclosed herein can also be designed such that the osteotomy guides and drill guides are configured to operate independently or can be configured to operate in combination. The drill guides and osteotomy guides can be modular so that different drill guides can be used for different patients. The drill guides and osteotomy guides can have conformal bone facing surfaces which are sized for patients of a given size category. The osteotomy guides can be designed to accommodate an array of potential surgical blades required by the surgeon. The shape of the drill guides (both in terms of the relative placement of the screw sites and the curvature of the bone facing surfaces) for the bone plate head and stem can correspond to an array of different size TPLO plates and bone screws used for final stabilization of the post-osteotomy bone segments.

While the specification has been described in detail with respect to specific embodiments of the invention, it will be appreciated that those skilled in the art, upon attaining an understanding of the foregoing, may readily conceive of alterations to, variations of, and equivalents to these embodiments. Any of the method steps discussed above can be conducted by a processor operating with a computer-readable non-transitory medium storing instructions for those method steps. The computer-readable medium may be memory within a personal user device or a network accessible memory. Although examples in the disclosure were generally directed to TPLO procedures for adjusting canine tibias, the surgical tools and methods disclosed herein area more broadly applicable to numerous other surgeries and medical applications including procedures in humans. Although examples in the disclosure were generally directed to the formation of screw holes in a patients bone which are intended to receive bone screws, cites which are used for any form of fastener that can attach a bone plate to a patient bone can be marked and applied using approaches disclosed herein. These and other modifications and variations to the present invention may be practiced by those skilled in the art, without departing from the scope of the present invention, which is more particularly set forth in the appended claims.

What is claimed is:

1. A set of tibial plateau leveling surgical implements, comprising:
a surgical guide having a drill guide and an osteotomy guide, wherein the osteotomy guide defines a potential site for a tibial plateau leveling osteotomy, and the drill guide has a distal guide screw hole and a proximal guide screw hole; and
a bone plate having a distal bone plate screw hole and a proximal bone plate screw hole;
wherein the drill guide and the bone plate are complementary in that: (i) the distal guide screw hole and the proximal guide screw hole are aligned with a planned pre-operative orientation of a planned distal bone screw location and a planned proximal bone screw location; and (ii) the distal bone plate screw hole and the proximal bone plate screw hole are aligned with a planned post-operative orientation of the planned distal bone screw location and the planned proximal bone screw location.

2. The set of tibial plateau leveling surgical implements of claim 1, wherein:
the drill guide is connected to the osteotomy guide and spans the potential site for the tibial plateau leveling osteotomy.

3. The set of tibial plateau leveling surgical implements of claim 1, wherein:
the surgical guide has a blade facing surface and a conformal bone facing surface; and
the conformal bone facing surface conforms to a standard tibial surface and is not patient specific.

4. The set of tibial plateau leveling surgical implements of claim 3, wherein:
the surgical guide is a reusable surgical guide and is made of metal.

5. The set of tibial plateau leveling surgical implements of claim 1, wherein the reusable surgical guide further comprises:
a proximal drill guide and a distal drill guide that form the drill guide; and
a unibody element spanning the potential site for the tibial plateau leveling osteotomy;
wherein the unibody element includes the osteotomy guide and at least one of: (i) the proximal drill guide; and (ii) the distal drill guide.

6. The set of tibial plateau leveling surgical implements from claim 1, further comprising:
a library of bone plates which includes the bone plate;
wherein the bone plate is paired with the reusable surgical guide for a potential tibial plateau angle rotation.

7. The set of tibial plateau leveling surgical implements from claim 6, further comprising:
a library of surgical guides which includes the surgical guide;
wherein the library of bone plates and the library of surgical guides form pairs of complementary bone plates and surgical guides for different potential tibial plateau angle rotations.

8. The set of tibial plateau leveling surgical implements from claim 1, further comprising:
a proximal drill guide and a distal drill guide that form the drill guide;
wherein: (i) the osteotomy guide forms an arc for the tibial plateau leveling osteotomy;
and (ii) the proximal drill guide includes an positioner implement that extends towards a center of the arc.

9. The set of tibial plateau leveling surgical implements from claim 1, further comprising:
a proximal drill guide and a distal drill guide that form the drill guide; and
a proximal alignment pin hole in the proximal drill guide that is more proximal than any screw hole in the proximal drill guide.

10. The set of tibial plateau leveling surgical implements from claim 1, further comprising:
a proximal drill guide and a distal drill guide that form the drill guide;
a proximal alignment pin hole in the proximal drill guide; and
a distal alignment pin hole in the distal drill guide;
wherein the proximal drill guide and the distal drill guide are part of a unibody element.

11. The set of tibial plateau leveling surgical implements from claim 1, further comprising:
a set of drill guide holes in the surgical guide;
a bone facing surface of the surgical guide; and
a blade facing surface of the surgical guide;
wherein: (i) the set of drill guide holes are aligned relative to the bone facing surface so that screws inserted into the set of drill guide holes are perpendicular to a sagittal plane; and (ii) the set of drill guide holes are off-normal from the blade facing surface.

12. The set of tibial plateau leveling surgical implements from claim 1, further comprising:
a set of at least one negative angle offset bone plates wherein each negative angle offset bone plate in the set of negative angle offsets has a head with a negative angle relative to the bone plate that is unique amongst the negative angle offset bone plates in the set of negative angle offset bone plates; and
a set of at least one positive angle offset bone plates wherein each positive angle offset bone plate in the set of positive angle offsets has a head that has a positive angle relative to the bone plate and that is unique amongst the positive angle offset bone plates in the set of positive angle offset bone plates.

13. The set of tibial plateau leveling surgical implements from claim 1, further comprising:
a bone facing surface of the surgical guide; and
a patellar tendon shield on the osteotomy guide that extends past the bone facing surface of the surgical guide.

14. The set of tibial plateau leveling surgical implements from claim 1, wherein:
the surgical guide has a blade facing surface and a conformal bone facing surface; and
the conformal bone facing surface conforms to a standard tibial surface and has a cut out at a standard popliteus muscle location.

15. The set of tibial plateau leveling surgical implements from claim 1, further comprising:
a rotation handle with a bone plate interface;
wherein the bone plate has a rotation handle interface to fix the bone plate to the rotation handle in combination with the bone plate interface.

16. The set of tibial plateau leveling surgical implements of claim 1, wherein:
a set of trajectories of a set of screws for the distal bone screw hole and proximal bone screw hole cause a post-operative bone compression that accounts for a kerf and that biases the post-operative bone compression to a trans-osteotomy side.

17. The set of tibial plateau leveling surgical implements of claim 1, further comprising:
a drill sleeve for insertion into a screw hole of the drill guide;
wherein the screw hole includes a first set of inward facing protrusions around a bone facing surface edge of the screw hole and a second set of inward facing protrusions around a blade facing surface edge of the screw hole.

18. A reusable surgical guide for a tibial plateau leveling osteotomy, comprising:
a blade facing surface with an osteotomy guide;
a conforming bone facing surface; and
a drill guide connected to the osteotomy guide that spans the potential site for the tibial plateau leveling osteotomy;
wherein the conformal bone facing surface conforms to a standard tibial surface and is not patient specific.

19. A surgical method for a tibial plateau leveling osteotomy, comprising:
obtaining an image of a patient bone to determine a patient tibial plateau angle;
selecting at least one of a surgical guide and a bone plate from a library based on the patient tibial plateau angle, wherein the surgical guide includes a conformal bone facing surface, and the conformal bone facing surface conforms to a standard tibial surface and is not patient specific;
applying the surgical guide to the patient bone; and
performing a tibial plateau leveling osteotomy of the patient bone using the surgical guide.

20. The surgical method for a tibial plateau leveling osteotomy of claim 19, wherein:
the image is a mediolateral radiograph.

21. The surgical method for a tibial plateau leveling osteotomy of claim 19, further comprising:
drilling a proximal screw hole and a distal screw hole in the patient bone using a drill guide of the surgical guide;
attaching a bone plate to the patient bone using: (i) a distal bone screw in a distal bone plate screw hole that is aligned with the distal screw hole; and (ii) a proximal bone screw in a proximal bone plate screw hole that is aligned with the proximal screw hole; and
rotating the patient bone;
wherein the drill guide and the bone plate are complementary in that: (i) the distal guide screw hole and the proximal guide screw hole are aligned with a planned pre-operative orientation of a planned distal bone screw location and a planned proximal bone screw location; and (ii) the distal bone plate screw hole and the proximal bone plate screw hole are aligned with a planned post-operative orientation of the planned distal bone screw location and the planned proximal bone screw location.

22. The surgical method for a tibial plateau leveling osteotomy of claim 21, wherein:
the drilling is conducted prior to the performing of the tibial plateau leveling osteotomy.

23. The surgical method for a tibial plateau leveling osteotomy of claim 21, wherein:
the library is a library of bone plates which includes the bone plate; and
the bone plate is complementary to the surgical guide in the library of bone plates for a tibial plateau leveling rotation.

24. The surgical method for a tibial plateau leveling osteotomy of claim 21, further comprising:
aligning a positioner implement with the intercondylar eminences of the patient bone while applying the surgical guide to the patient bone;
wherein: (i) the surgical guide includes an osteotomy guide and a proximal drill guide;
(ii) the osteotomy guide forms an arc for the tibial plateau leveling osteotomy; (iii) the proximal drill guide includes the positioner implement; (iv) the positioner implement extends towards the center of the arc; and (v) the positioner implement is more proximal than any screw hole in the proximal drill guide.

25. The surgical method for a tibial plateau leveling osteotomy of claim 24, further comprising:
placing a proximal alignment pin in a proximal alignment pin hole in a proximal drill guide of the surgical guide after applying the surgical guide to the patient bone;
wherein the proximal alignment pin hole is more proximal than any screw hole in the proximal drill guide.

26. The surgical method for a tibial plateau leveling osteotomy of claim 25, further comprising:
aligning a distal alignment pin hole in distal drill guide of the surgical guide on a center of the patient bone after placing the proximal alignment pin;
placing a distal alignment pin in the distal alignment pin hole after aligning the distal alignment pin hole; and
drilling a proximal screw hole and a distal screw hole in the patient bone using the drill guide after placing the distal alignment pin.

27. The surgical method for a tibial plateau leveling osteotomy of claim 23, wherein:
the bone plate was selected from the library;
the bone plate is an offset bone plate; and
the offset plate is selected from: (i) a set of at least one negative angle offset bone plates wherein each negative angle offset bone plate in the set of negative angle offsets has a head with a negative angle relative to the bone plate; and (ii) a set of at least one positive angle offset bone plates wherein each positive angle offset bone plate in the set of positive angle offsets has a head that has a positive angle relative to the bone plate.

28. The surgical method for a tibial plateau leveling osteotomy of claim 19, further comprising:
attaching the bone plate to the patient bone using a set of bone screws;
attaching a rotation handle to the bone plate, wherein the bone plate has a rotation handle interface to fix the bone plate to the rotation handle in combination with the bone plate interface; and
rotating, after performing the tibial plateau leveling osteotomy, the patient bone using the rotation handle.

29. The surgical method for a tibial plateau leveling osteotomy of claim 19, further comprising:
obtaining a craniocaudal radiograph image of the patient bone; and
selecting lengths for the set of bone screws using the craniocaudal radiograph.
